(19) United States
(12) Patent Application Publication
(10) Pub. No.: US 2017/0095494 A1
(43) Pub. Date: Apr. 6, 2017

(54) Process for the Extraction of Strawberry P-Coumaroyl Hexose and/or Polyphenols, Extract and Uses Thereof

(71) Applicants: Nutra Canada Inc., Champlain (CA); Universite Laval, Quebec (CA)

(72) Inventors: Sebastien LEONHART, Champlain (CA); Andre GOSSELIN, Champlain (CA); Andre MARETTE, St-Nicolas (CA); Genevieve PILON, Sainte-Catherine de La Jacques-Cartier (CA); Paul ANGERS, Quebec (CA); Yves DESJARDINS, Quebec (CA); Stephanie DUDONNE, Quebec (CA); Pascal DUBE, Quebec (CA)

(21) Appl. No.: 15/126,435

(22) PCT Filed: Mar. 16, 2015
(86) PCT No.: PCT/CA2015/050194

§ 371 Date: Sep. 15, 2016

Related U.S. Application Data
(60) Provisional application No. 61/954,004, filed on Mar. 17, 2014.

(51) Int. Cl. A61K 31/7034; A61K 36/73

(52) U.S. Cl. CPC A61K 31/7034; A61K 36/73; A61K 2236/00

(57) ABSTRACT

A composition of purified and biologically active p-coumaroyl hexose and/or polyphenols is provided by separation from strawberry's pulp by a method of extraction and purification using a hydrophobic adsorbent using GRAS solvent. Also provided is the use of the composition.

### FIELD OF THE INVENTION

The present invention relates to a process for the extraction of p-coumaroyl hexose and/or polyphenols or other anti-oxidant compounds from strawberry, to compositions thus obtained and their use as anti-diabetic compounds.

### BACKGROUND OF THE INVENTION

Recent research has shown that strawberries puree and extracts exhibit potent biological properties attributable to the presence of polyphenols. These hydrolysable tannins are present in high levels in strawberries and include ellagintanins, anthocyanins, proanthocyanidins and flavonoids. Strawberry tannins have been identified as an active antioxidant compounds responsible for their biological activity. Unfortunately, there are no methods currently available for rapid and large scale production of these polyphenols without the use of strong organic solvents. This invention proposes a method to produce extracts rich in polyphenols from strawberry and outlines their uses.

Ellagitanins are the predominant strawberry polyphenols, and are responsible for the high antioxidant activity of strawberries. Previous methods of isolating strawberry and other berries' polyphenols include time consuming preparative high-performance liquid chromatographic (HPLC) and/or column chromatographic methods in conjunction with the use of strong organic solvents. In view of the commercial interest in these compounds, the development of inexpensive, high throughput methods of purification are of particular interest, particularly in conjunction with the use of solvents that are safe for the food industry, especially solvent labelled as GRAS (Generally Recognized as Safe).

Also, p-coumaric acid is found at various concentrations in some polyphenol-rich supplements. This phenolic acid was identified in high concentrations in rat plasma after polyphenol ingestion (Dudonné et al, 2014). However, p-coumaroyl hexose has not yet been disclosed and its physiological role has not yet been established with certainty.

### SUMMARY OF THE INVENTION

According to a first aspect, the invention provides a composition substantially enriched in p-coumaroyl hexose as well as methods for producing such a composition and its uses.

According to a second aspect, the invention provides a composition of enriched and biologically active polyphenols, specifically including ellagitanins, proanthocyanindins, anthocyanin and flavonoids, as well as methods for producing such composition and its uses.

In a third aspect, p-coumaroyl hexose and polyphenols are separated from strawberry natural products by a method of extraction and purification using a solvent extraction process and purification with chromatography on hydrophobic adsorbents.

In a particular aspect, the invention provides a process for the extraction of p-coumaroyl hexose and/or polyphenols from strawberry comprising the steps of:
obtaining a processed strawberry pulp;
suspending said pulp in ethanol/water to obtain an ethanolic extract;
evaporating said ethanol and resuspending extract in water to obtain an aqueous extract;
submitting said aqueous extract to a hydrophobic chromatography column;
eluting said column with ethanol/water gradient to obtain a solution enriched in p-coumaroyl hexose and/or polyphenols; and
optionally drying said solution to yield a powder highly concentrated in total p-coumaroyl hexose and/or polyphenols.

Particularly, the present invention provides a purified strawberry extract composition comprising a concentration of at least about 500 ppm of p-coumaroyl hexose. More particularly, the composition further comprises a physiologically acceptable excipient and/or a conservative agent.

In an alternative aspect, the invention provides a purified strawberry extract comprising about 5 to 60% polyphenols.

In a particular aspect, the invention provides an enriched strawberry extract, comprising about 30-40% ellagitanins; 20-30 proanthocyanidins; 5 to 10% pelargonidin; and 5-10% quercetin.

In a particular aspect, the invention provides an enriched strawberry extract in the form of a powder.

Particularly, the invention provides an enriched strawberry extract wherein the strawberry variety is Authentique Orléans.

Fractions comprising strawberry polyphenols and/or p-coumaroyl hexose are useful for a variety of applications, including pharmaceutical, nutraceutical, cosmetic, and food uses. Particularly, the invention provides a method for the treatment of inflammation comprising administering an effective amount of a strawberry extract as defined herein. Alternatively, the invention provides the use of a strawberry extract as defined herein for the treatment of inflammation.

### DETAILED DESCRIPTION OF THE INVENTION

### Description of the Figures

FIG. 1. Ultra-high pressure liquid chromatography (UPLC) fingerprint of Authentique Orléans strawberry.

FIG. 2. High pressure liquid chromatography (HPLC) fingerprint of Authentique Orléans' PACs.

FIG. 3. Effect of strawberry fraction on basal and stimulated glucose transport in L6 muscle cells (n=6).

FIG. 4. Effect of strawberry fraction on nitrite production in FAO hepatocytes (n=3).

FIG. 5. Effect of strawberry fraction on basal nitrite production in J774 macrophages (n=6).

FIG. 6. Concentration of coumaric acid and derivatives in Authentique Orléans strawberry.

FIG. 7. Structure of p-coumaroyl hexose.

FIG. 8. A) Glucose disposal rate (GDR) (mg·kg⁻¹¹·min⁻¹) and B) insulin sensitivity (M/I) (mg·kg⁻¹·min⁻¹·pmol⁻¹) before (Pre) and after (Post) the 6-week experimental period. Values are means±standard error of the mean (SEM) represented by vertical bars, n=39. * P<0.05, ** P<0.01. P values refer to comparisons between the variations of the two groups with the baseline M/I as covariate. C) Responses of plasma C-peptide at 0, 15, 30, 60 and 120 min during the OGTT before (Pre) and after (Post) intake of Polyphenol and Placebo. Dotted line−circles=Polyphenol Pre values, continous line−circles=Polyphenol Post values, dotted line−triangles=Placebo Pre values, continous line−triangles=Placebo Post values. Values are means±standard error of the mean (SEM) represented by vertical bars, n=41. P=0.002. D) Repeated measures ANOVA for C-peptide (pmol·L⁻¹) over time during the OGTT, expressed as mean variations (Post values−Pre values) for C-peptide concentrations. Circles=Polyphenol, Triangles=Placebo. Positive incremental area under the curve (IAUC), respectively E) for the 120 minutes and F) for the first 30 minutes of the OGTT for C-peptide concentrations (pmol·L⁻¹·min⁻¹). Values are means±standard error of the mean (SEM) represented by vertical bars, n=41. * P<0.05, ** P<0.01. P values refer to comparisons between the variations of the two groups.

FIG. 9. Effects of p-coumaroyl hexose and p-coumaric acid on basal and insulin-stimulated glucose uptake.

### ABBREVIATIONS AND DEFINITIONS

### Abbreviations

EPP: extractable polyphenols; NEPP: non-extractable polyphenols; PAC: Proanthocyanindins; PP: polyphenols; SPE: solid phase purification.

### DEFINITIONS

The term “about” as used herein refers to a margin of + or −10% of the number indicated. For sake of precision, the term about when used in conjunction with, for example: 90% means 90%+/−9% i.e. from 81% to 99%. More precisely, the term about refer to + or −5% of the number indicated, where for example: 90% means 90%+/−4.5% i.e. from 86.5% to 94.5%.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

### Detailed Description of Particular Embodiments

### Extraction of Polyphenols

In a particular aspect, the invention provides a process for the extraction of strawberry polyphenols comprising the steps of:
obtaining a processed strawberry pulp;
suspending said pulp in ethanol/water to obtain an ethanolic extract;
submitting said ethanolic extract to a hydrophobic chromatography column (such as XAD-7 or DUAS 2525);
eluting said column with ethanol/water to obtain a solution enriched in polyphenols; and
optionally spray drying said solution to yield a powder highly concentrated in total polyphenols.

In a particular embodiment of the invention, the pulp from strawberry fruits or leaves is processed physically or enzymatically to obtain a paste, pulp or paste. The resulting paste is suspended in ethanol/water for extraction at a concentration of about 50%, incubated at room temperature for about 5 min to about 3 hours and the solids are separated from the solvent by filtration. The resulting ethanolic extract is further submitted to a hydrophobic chromatography column on XAD-7 or DUAS 2525 to enrich strawberry polyphenols from the ethanolic extract. The polyphenols are adsorbed on the columns, and obtained by elution with a gradient of about 96 to about 50% ethanol (cleansing, gravity evacuation, and vacuum aspiration of fluid from the column), the adsorbed polyphenols are eluted from the column. The resulting solution is spray dried to yield a powder of highly concentrated total polyphenols (between about 10% up to 60% polyphenols) comprising a high percentage (e.g., 30 to 40%) of ellagitanin, as well as a significant percentage of proanthocyanidin (20-30%) and anthocyanin about 7.5% (mostly pelargonidin) and flavonoids 5-6% (mostly quercetin).

### Processing of Pulp

The berries or leaves are physically disrupted by blending, grinding, crushing, pressing or sonicating in order to obtain a puree that is suitable for suspension in an extraction solvent. Alternatively, the pulp may be subjected to enzymatic treatment including but not limited extractase, pectinase and the likes.

### Suspension in Ethanol/Water and Extraction

The thus obtained strawberry puree is then exposed to the solvent and incubated for about 5 min to about 3 hours at about 15° C. to about 30° C., particularly about 22° C.

### Purification

Hydrophobic adsorbent resins are used to purify polyphenols from an aqueous strawberry extract. The aqueous solution comprising the polyphenols is applied to a polymeric adsorbent column, which is then washed with an aqueous buffer to remove unbound material. The polyphenols of interest bind to the resin and are eluted with GRAS solvent, particularly with ethanol or a mixture or gradient of ethanol/water. After adsorption, the resin is subjected to cleansing, gravity evacuation, and vacuum aspiration of fluid.

The resin has a surface to which the polyphenols are adsorbed. A preferred class of adsorptive resins are polymeric cross-linked resins composed of styrene and divinylbenzene such as, for example, the AMBERLITE series of resins. It is preferred to use commercially available, FDA-approved, styrene-divinylbenzene (SDVB) cross-linked copolymer resin, (e.g., AMBERLITE XAD-7 or DUETA DUAS 2525). Thus, in one embodiment, AMBERLITE XAD-7, commercially available from Rohm and Haas Company, are or DUAS 2525 available from DUETA Natural Products Industry can be used as the resin. These resins are a non-ionic hydrophobic, cross-linked polystyrene divinyl benzene adsorbent resin. Particularly, AMBERLITE XAD-7 has a macroreticular structure, with both a continuous polymer phase and a continuous pore phase. In a particularly preferred embodiment, the resin used in the present invention has a particle size ranging from 100-200 microns.

Other adsorbents, such as those in the AMBERLITE XAD adsorbent series which contain hydrophobic macroreticular resin beads, with particle sizes in the range of 100-200 microns, are also be effective in the methods of the present invention. Moreover, different variations of the AMBERLITES, such as the AMERCHROM CG series of adsorbents, used with particle sizes in the range of 100-200 microns, may also be suitable for use in the present invention. The AMBERLITE XAD-7 is particularly suitable since it can be re-used many times (over 100 times). However, it is contemplated that for food, the use of governmentally-approved resins in the present invention may be considered important and/or desirable. Various geometries may be used for the purification, including batch adsorption, column chromatography, and the like, as known in the art.

The resins are washed, e.g. with water or an aqueous buffer to remove unbound material from the extract. GRAS solvents are used to remove the adsorbed polyphenols. Preferred GRAS solvents are water and ethanol(ethyl alcohol) since they are approved for food use. Typically the ethanol is azeotroped with water; however, absolute ethanol can be used.

The eluted polyphenols are substantially purified relative to the starting material, and may be further purified, e.g. by chromatography, etc., or may be directly used in formulations of interest. The final composition may be concentrated, filtered, dialyzed, etc., using methods known in the art.

### Drying

It will be appreciated that any suitable method may be employed for dewatering or drying the eluted polyphenol solution such as heating or vacuum drying to a powder. Typically, a powder in accordance with the present invention is prepared by lyophilization, freeze-drying or spray drying. More particularly, the polyphenol solution is spray-dried.

### Compositions

Compositions of interest are obtained from the above purification process. The compositions comprise between about 5% to about 60% polyphenols, particularly, between 10% and 50%, more particularly between 30% and 40%, and may be provided as a powder, in solution, e.g. in water or aqueous buffer, ethanol, etc. Such compositions may comprise usually at least about 25% ellagitannins as either weight/volume or percentage of weight; at least about 15% proanthocyanidines; at least about 5% anthocyanidins; and at least about 5% flavonoids.

Compositions of interest comprise a purified strawberry extract having an ORAC value of at least about 1000 Trolox equivalent (μM/g), particularly at least about 1200, more particularly at least about 1500, most particularly at least about 1700 Trolox equivalent (μM/g).

Included in the compositions of the invention are extracts comprising about 30 to 40% ellagitanins. Also included in the compositions of the invention are extracts comprising proanthocyanins at a concentration of about 20 to 30%. In another embodiment, compositions are provided comprising anthocyanins and pelargonidin at a concentration of about 5% to 10%. In another embodiment, compositions are provided comprising quercetin at a concentration of about 5 to 10%.

Also included in the compositions of the invention are extracts comprising p-coumaroyl hexose in a concentration of at least about 500 ppm of dry matter. Also included in the compositions of the invention are extracts comprising p-coumaroyl hexose at a concentrations of at least 600, at least 800, at least 900, or at least 1000 ppm. In another embodiment, compositions are provided comprising p-coumaroyl hexose at a concentrations of at least 2000, at least 3000, at least 4000, at least 5000 or at least 6000 ppm of dry matter. As well, these extracts may also comprise other polyphenols as described herein.

The extracts thus obtained may be used in biological studies, for pharmaceutical uses; in the preparation of tinctures, cosmetics and other therapeutic formulae, as food additives, in the nutraceutical industry; and the like. The isolated mixtures of polyphenols: ellagitannins, proanthocyanins, anthocyanins and/or anthocyanidins can also be tableted or used as capsules, soft gels and the likes and used as a natural nutraceutical/dietary supplement. In general, the tablets, capsules, soft gels etc. provide a daily dose of the tannins. The amount of the polyphenols can be adjusted by isolating the individual compounds and blending them together. The tablets, capsules, soft gels etc. may comprise the natural mixture of the polyphenols and/or p-coumaroyl hexose that are isolated by the resin, optionally in admixture with a physiologically acceptable excipient and/or a conservative agent.

### Use of Polyphenols

The extract thus obtained may be used in the preparation of tinctures, cosmetics and other therapeutic formulae, as food biopreservatives, in the nutraceutical industry; and the like. The compositions also find use as a source of polyphenols, for use in in vitro and in vivo biological studies. The compounds thus isolated are reported to have antioxidant and anti-inflammatory activity. The tannins obtained by the methods of the invention may be used to formulate pharmaceuticals, nutraceuticals, herbal medicines, food additive, cosmetics, beverages, etc.

For therapeutic applications, the polyphenols are administered to a mammal in a physiologically acceptable dosage form, including those that may be administered to a human orally, etc. as a bolus.

The extracts of the invention may be provided as a composition with a pharmaceutically acceptable carrier. Such dosage forms encompass physiologically acceptable carriers that are inherently non-toxic and non-therapeutic. Examples of such carriers include ion exchangers, soft gels, oils, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of tannins include polysaccharides such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The extracts will typically be formulated in such vehicles at a concentration of about 0.1 μg/ml to 100 μg/ml and higher.

Nutraceutical formulations of interest include foods for veterinary or human use, including health food bars, drinks and drink supplements, and the like. These foods are enhanced by the inclusion of a biologically active extract of the invention. For example, in the treatment of chronic inflammatory diseases, such as arthritis, the normal diet of a patient may be supplemented by a nutraceutical formulation taken on a regular basis.

Particularly, the extract of the present invention may be formulated for the use against, prevention or treatment of type-2 diabetes or metabolic syndrome. More particularly, the extract may be administered in pre-diabetic subjects for reversing insulin resistance and improving glucose homeostasis.

For cosmetic formulations, the compositions of the invention may optionally comprise skin benefit materials. These include estradiol; progesterone; pregnanalone; coenzyme Q10; methylsolanomethane (MSM); copper peptide (copper extract); plankton extract (phytosome); glycolic acid; kojic acid; ascorbyl palmitate; all-trans-retinol; azaleic acid; salicylic acid; broparoestrol; estrone; adrostenedione; androstanediols; etc. The steroids will generally present at a concentration of less than about 2% of the total by weight of the composition, while the other skin benefit materials may be present at higher levels, for example as much as 10 to 15%.

The compositions of the invention may comprise a cosmetically acceptable vehicle to act as a diluant, dispersant or carrier, so as to facilitate its distribution when the composition is applied to the skin. Vehicles other than or in addition to water can include liquid or solid emollients, solvents, humectants, thickeners and powders.

The cosmetically acceptable vehicle will usually form from 0.1%, or 5% to 99.9%, preferably from 25% to 80% by weight of the composition, and can, in the absence of other cosmetic adjuncts, form the balance of the composition.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXPERIMENTAL

### Example 1—Materials and Methods

### Chemicals

Gallic acid and Amberlite XAD7 were obtained from Sigma-Aldrich (USA). Folin-Ciocalteu's phenol reagent was purchased from Merck (USA). All other solvents used were of analytical grade and purchased from local distributors.

### Raw Materials

Agricultural Conditions and Soil Characteristics. All commodities used in these studies were grown under controlled conditions and supplied by Les Fraises de l'\hat{l}le d'Orléans (St Laurent de l'île d'Orléans, Qc). Sample selection was based upon matched crop availability from this farm. The strawberry variety was Authentique Orléans. Records were kept on agricultural conditions, soil type, irrigation source, and chemical applications. The fruits are harvested at three dates during the production period (Jun. 28, Jul. 4 and Jul. 21, 2012). Three (3) samples from each date were grinded and freeze-dried for 3 days until dryness.

### Extraction

Half a gram of freeze-dried fruit was placed in a capped centrifuge tube; 20 mL of ethanol/water (50:50, v/v; pH 2) acidified with 2 N HCl was added, and the tube was sonicated for 30 minutes. The sample was centrifuged at 3500 rpm for 5 min and the supernatant recovered. The extractable polyphenols (EPP) were quantified by spectrophotometer (Folin) and reverse-phase UPLC-MS and EPP content by BL-DMAC and by normal-phase HPLC. The solvents were removed from the liquid extract by evaporation in a rotary evaporator at 45° C. The extract was then dissolved in 10 ml of water before being fractionated on the resin by solid phase purification (SPE).

### Solid Phase Purification

The resin (XAD7-HP) was soaked in distilled water, and then loaded into the column. After being rinsed, the crude extract was loaded onto the column. Impurities were eluted with distilled water. Ten mL of the eluate fractions were collected until no more soluble sugars could be detected. The polyphenols were then eluted with aqueous ethanol at a concentration between 96 and 40%. To monitor the elution profile, fractions of 10 ml were collected and analyzed. The richest polyphenols fraction of eluent was concentrated by rotary evaporation at 50° C. The concentrated purified fraction was freeze dried to obtain a product in the form of a dry powder. The powder thus obtained was maintained at 18° C. until analysis.

### Example 2—Analysis of Purified Fraction

### Analysis

The total phenolic content of the purified fraction was determined with Folin-Ciocalteu assay using gallic acid as a standard (Singleton et al., Methods in Enzymol. 1999; 299; 14) after the powder was dissolved in ethanol or methanol. Total proanthocyanidins content was analyzed with 4-dimethylaminocinnamaldehyde (DMAC) colorimetric method using a dimer of PAC A2 as a standard (Prior et al. J Sci Food Agric 2010; 90; 1473-1478).

### UPLC Analysis

Analyses of EPP, and NEPP in fractions were performed by UPLC. The UPLC system was equipped with a binary gradient pump, a sample injector, a column oven, a photodiode array detector, and a degassing system and driven by Waters Empower software. Two microliters of the diluted fraction (in what?) was injected onto the UPLC system and separated by an C-18 Zorbax RRHD Eclipse Plus 2.1×100 mm, 1.8 μm. The binary system phases were (A) water with 0.1% formic acid and (B) acetonitrile, with a flow rate of 0.2 mL/min, giving a maximum back pressure of 5600 psi, which is within the capabilities of the UPLC. The 69 min gradient was as follows: 0-6 min, 95-88% A (linear); 6-12 min, 88-85% A; 12-24 min, 85-75% A; 24-33 min, 75-70% A linear; 33-39 min, 70-65% A; 39-51 min, 65-40%; 51-61.8 min, 40-05% A nonlinear; 61.8-62.4 min, 5-95% A linear and 62.4-69, 95% A re-equilibration time. Channel 1 detection was performed at 280 nm, and a spectrum was recorded at 210-650 nm to aid identification. Channel 2 detection was performed at 360 nm, and a spectrum was recorded at 310-410 nm. The UPLC fingerprint from the strawberry fraction is presented in FIG. 1.

### HPLC Analysis

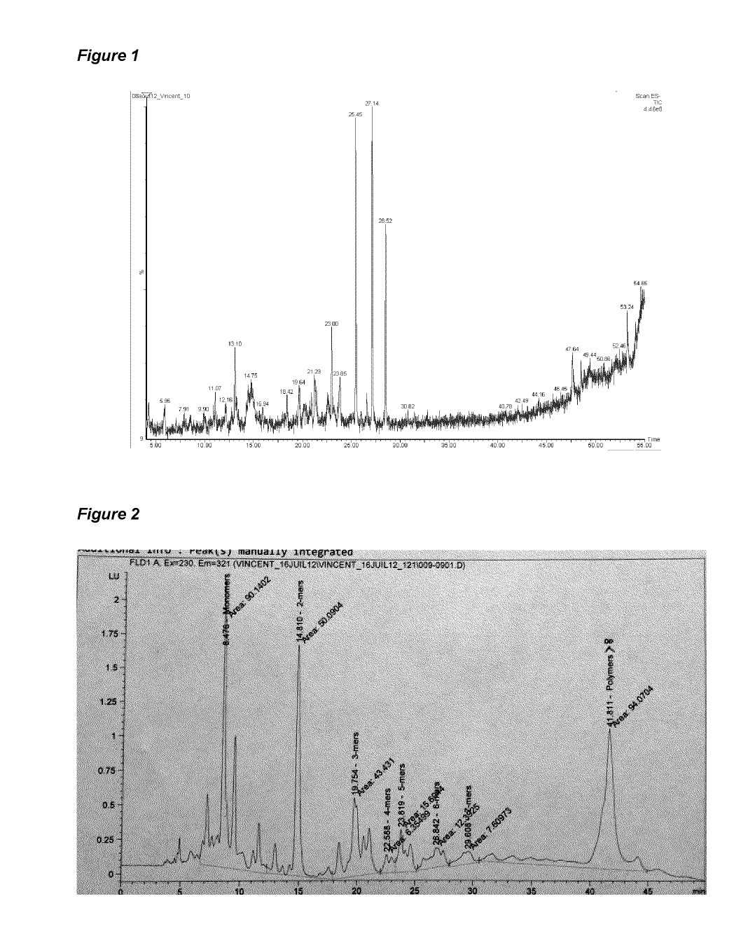

Samples were analyzed using a HPLC equipped with LC-20AD pumps, SIL-20AC auto sampler, and a CTA-20A Column Oven coupled to SPD-M20A Photodiode Array (Shimadzu), RF-10AXL Fluorescence (Shimadzu). All samples were filtered through 0.45 micrometers polypropylene filters prior to HPLC analysis. Procyanidin analysis was performed according to an adapted method from Taylor et al. (2010). A normal phase 4.6×250 mm Develosil Diol column with a 5-μm size (Phenomenex) was connected to a 4×3 mm Cyano Security-Guard column (Phenomenex) for the analysis. Solvents and samples were filtered through 0.45 um polypropylene filters. Separation of strawberry-fraction procyanidins was achieved using a linear gradient from 0% to 40% B, in 35 min; 40% to 100% B, in 40 min; 100% to 100% isocratic B, in 45 min; and 100% to 0% B, in 50 min. The column was reequilibrated for 5 min between samples. The flow rate was set at 0.8 mL/min. Solvent A was 2% acetic acid in acetonitrile and B was 95:3:2 methanol/water/acetic acid. The injection volume was 5 μL, and column temperature was kept at 35° C. Fluorescence of the procyanidins was monitored at excitation and emission wavelengths of 230 and 321 nm with the fluorescence detector. The fluorescence detector was set to low sensitivity with a gain of 4× for the entire run. Each sample was run in duplicate, and the order of injection was randomized. Commercially available catechin (0.1 g) was dissolved in 100 mL of 70.0:29.5:0.5 acetone/water/acetic acid and a series of dilutions were prepared to generate a standard curve (r2>0.99). Each individual procyanidin peak in all strawberry-fraction samples tested contained a peak area that was within the catechin standard curve. Total procyanidins from all strawberry-fraction were expressed as catechin equivalents by weight. Calculation of total procyanidins, as well as, individual procyanidins grouped by their degree of polymerization was reported based on the calibration curve. The strawberry-fraction HPLC fingerprint is presented at FIG. 2.

### Mass Spectrometric Analysis.

Accurate mass and fragmentation pattern information was obtained using a Micromass Q-TOF II hybrid mass spectrometer equipped with an electrospray ionization (ESI) ion source. Major experimental parameters were as follows: nebulizer gas (nitrogen) 650 L/h; auxiliary gas, 250 L/h; cone gas, 15 L/h; source block temperature, 120° C.; nebulizer temperature, 350° C.; time-of-flight potential, 9.1 kV; multichannel plate potential, 2200 V. In negative mode 3 kV needle voltage and 39 V cone voltage were used. Mass range was 20-1974. Mass spectrometric analysis was performed in the ESI-mode and set up in the selected ion recording (SIR) mode. The system was collection by Masslynx™ V 4.1 software (Micromass, Manchester, UK). The analytes were assayed by quantifying the [M-H]− ions of specific m/z (89 different molecules). Scan of the standards (gallic acid, epicatechin, catechin, chlorogenic acid, p-coumaric acid, quercetin, quercetin-glucoside, ellagic acid, dimer of PAC A2, protocatechuic ac.) is shown in Table 1.

TABLE 1
Retention time, inonic mass spectromety, maximum wavelength by diod
array detector of polyphenols detected by UPLC.

| Name | R.T | M − H | ms/ms (negative) | | | | | DAD | | | REF |
|---|---|---|---|---|---|---|---|---|---|---|---|
| Gallic acid | 5.1 | 169 | 125 | | | | | 219 | 272 | | (Std Sigma) |
| Protocatechuic acid | 8.4 | 153 | 109 | 80 | | | | 218 | 259 | 293 | (Std Sigma) |
| m-Coumaric acid | 9.3 | 163 | 119 | | | | | | | | (Fang et al., |
| | | | | | | | | | | | 2002) |
| p-Coumaric acid 4- | 9.3 | 325 | 163 | | | | | | | | (Maatta- |
| O-glucoside | | | | | | | | | | | Riihinen et al., |
| | | | | | | | | | | | 2004) |
| 5-Caffeoylquinic acid | 11.8 | 353 | 191 | 179 | 161 | | | 245 | 319 | | (Std Sigma) |
| 1-Caffeoylquinic acid | 11.9 | 353 | 191 | 179 | | | | | | | (Clifford et al., |
| | | | | | | | | | | | 2003) |
| o-Coumaric acid | 12 | 163 | 119 | | | | | | | | (Fang et al., |
| | | | | | | | | | | | 2002) |
| 2,3 dihydroxybenzoic | 12.1 | 153 | 109 | 80 | | | | 216 | 327 | | (Std Sigma) |
| (+)-Catechin | 12.3 | 289 | | | | | | | | | (Std Sigma) |
| p-Coumaroyl | 12.4 | 325 | 267 | 205 | 187 | 163 | 145 | | | | (Aaby et al., |
| glucose | | | | | | | | | | | 2007) |
| p-hydroxybenzoic | 12.7 | 137 | 93 | | | | | | | | (Fang et al., |
| acid | | | | | | | | | | | 2002) |
| Ac vanillic | 13.6 | 167 | 152 | 123 | 108 | | | | | | (Std Sigma) |
| p-coumaroyl-ester | 13.9 | 355 | 295 | 217 | 193 | | | | | | (Del Rio et al., |
| | | | | | | | | | | | 2004) |
| 3-Caffeoylquinic acid | 14.2 | 353 | 191 | 179 | | | | | | | (Clifford et al., |
| | | | | | | | | | | | 2003) |
| 4-p-Coumaroylquinic | 15.6 | 337 | 173 | | | | | | | | (Del Rio et al., |
| acid | | | | | | | | | | | 2004) |
| (−)-Epicatechin | 15.8 | 289 | | | | | | 210 | | | std Sigma |
| Bis galloyl HHD- | 16.4 | 783 | 634 | | | | | | | | (Aaby et al., |
| glucose | | | | | | | | | | | 2007) |
| Naringenin | 16.7 | 271 | 151 | | | | | | | | (Pulcini et al., |
| | | | | | | | | | | | 2006) |
| Myricetin | 16.9 | 317 | 271 | 317 | 151 | | | | | | (Pulcini et al., |
| | | | | | | | | | | | 2006) |
| 5-p-Coumaroylquinic | 18.8 | 337 | 191 | 173 | | | | | | | (Fang et al., |
| acid | | | | | | | | | | | 2002) |
| p-Coumaric acid | 19.3 | 163 | 119 | 89 | | | | 230 | 307 | | (Std Sigma) |
| Myricetin 3-O- | 19.5 | 479 | 315 | 179 | | | | 267 | 308 | | (Singh et al., |
| galactoside | | | | | | | | | | | 2011) |
| Myricetin-3-a- | 21.5 | 449 | 316 | 295 | 208 | | | | | | (Singh et al., |
| xylopyrannoside | | | | | | | | | | | 2009) |
| Sinapic acid | 21.6 | 223 | 164 | 148 | | | | 323 | | | (Std Sigma) |
| Myricetin 3-O- | 22.0 | 449 | 317 | 179 | | | | | | | (Singh et al., |
| arabinoside | | | | | | | | | | | 2009) |
| Ellagic acid | 22.6 | 301 | | | | | | | | | (Std Sigma) |
| Quercetin 3-O- | 22.9 | 463 | 301 | | | | | 210 | 269 | | (Std Sigma) |
| glucoside | | | | | | | | | | | |
| Dimere A | 23.6 | 575 | 449 | | | | | | | | (Std Sigma) |
| Quercetin-3-α- | 23.8 | 433 | 300 | 343 | 179 | | | 350 | | | |
| xylopyranoside | | | | | | | | | | | |
| Benzoic acid | 23.9 | 121 | 77 | | | | | | | | (Lee et al., |
| | | | | | | | | | | | 2008) |
| Quercetin-3-α- | 24.6 | 433 | 300 | 273 | 179 | | | 350 | | | (Vvedenskaya |
| arabinopyranoside | | | | | | | | | | | and Vorsa, |
| | | | | | | | | | | | 2004) |
| Quercetin-3-α- | 25.3 | 433 | 300 | 343 | 179 | | | 350 | | | (Vvedenskaya |
| arabinofuranoside | | | | | | | | | | | and Vorsa, |
| | | | | | | | | | | | 2004) |
| Kaempferol-p- | 25.5 | 447 | 285 | 178 | | | | 217 | | | (Buendil a et |
| coumaroyl | | | | | | | | | | | al., 2009) |
| Methoxyquercetin | 25.8 | 447 | 301 | 285 | | | | 210 | 351 | | (Buendil a et |
| pentosid | | | | | | | | | | | al., 2009) |
| Myricetin 3-O- | 26.1 | 463 | 315 | 179 | | | | | | | (Silva et al., |
| rhamnoside | | | | | | | | | | | 2005) |
| Phloridzin | 28.3 | 435 | 273 | 255 | 167 | 179 | | | | | (Std Sigma) |
| 3β- | 28.3 | 447 | 315 | | | | | | | | (Vvedenskaya |
| Methoxyquercetin-3- | | | | | | | | | | | and Vorsa, |
| α-xylopyranoside | | | | | | | | | | | 2004) |
| Kaempferol 3-O- | 28.9 | 431 | 285 | | | | | | | | (Han et al., |
| rhamnoside | | | | | | | | | | | 2005) |
| Quercetin | 34.3 | 301 | 151 | 179 | 273 | | | 210 | 254 | 370 | (Std Sigma) |
| Kaempferol | 41.5 | 285 | | | | | | | | | (Std Sigma) |
| Rhamnetin | 42.2 | 315 | 300 | 151 | | | | | | | (Monagas et |
| | | | | | | | | | | | al., 2010) |

For EPP, 20 μL of aqueous-organic extracts was injected onto a Phenomenex (Torrance, Calif.) 5 μm Luna silica (2), 100 A column (25×4.6 mm) at 37° C. HPLC column coupled to an Agilent 1100HPLC system with fluorescence detector and analyzed according to the method described by (Gu et al., 2004). with the additional relative fluorescence response data published by Prior (Prior and Gu, 2005). pure standards of epicatechin, and procyanidin dimers A2 were run under the same normal-phase HPLC conditions.

### Results

The concentration of total polyphenols increased from 1.2% in the dry powder of Authentique Orléans to up to 42% in the dried fractions after solid phase purification. PACs and UPLC profile remain unchanged. The concentration of polyphenols increased by 33 times on average after purification (Table 2).

TABLE 2
Concentration of different polyphenols in the primary extract and in the
purified fraction of “Authentique Orléans”.

| | Extract | | | Purified fraction | | |
|---|---|---|---|---|---|---|
| | (mg/g) | | | (mg/g) | | |
| Trimère A (2) | 0.002 | 0.000 | 0.001 | 0.048 | 0.006 | 0.023 |
| (−)-Epicatechin | 0.050 | 0.037 | 0.052 | 1.419 | 1.439 | 1.435 |
| (+)-Catechin | 1.373 | 0.751 | 1.424 | 39.072 | 29.353 | 39.680 |
| 1-Caffeoylquinic acid | 0.000 | 0.001 | 0.002 | 0.010 | 0.028 | 0.049 |
| 2,3-dihydroxybenzoic | 0.000 | 0.000 | 0.002 | 0.005 | 0.004 | 0.056 |
| 3,4-DHPEA-EDA | 0.001 | 0.000 | 0.000 | 0.015 | 0.004 | 0.008 |
| 3-Caffeoylquinic acid | 0.000 | 0.001 | 0.000 | 0.010 | 0.021 | 0.010 |
| 4-Caffeoylquinic acid | 0.001 | 0.000 | 0.001 | 0.036 | 0.011 | 0.016 |
| 4-Hydroxybenzoic acid 4-O-glucoside | 0.220 | 0.181 | 0.211 | 6.266 | 7.073 | 5.889 |
| (hydroxybenzoyl hexose) | | | | | | |
| 4-mere (1) | 0.000 | 0.001 | 0.001 | 0.010 | 0.039 | 0.020 |
| 4-mere (2) | 0.001 | 0.001 | 0.001 | 0.017 | 0.034 | 0.022 |
| 4-mere (3) | 0.000 | 0.000 | 0.000 | 0.002 | 0.009 | 0.011 |
| 4-p-Coumaroylquinic acid | 0.001 | 0.000 | 0.000 | 0.017 | 0.017 | 0.011 |
| 5-Caffeoylquinic acid (Chlorogenic acid) | 0.006 | 0.000 | 0.005 | 0.177 | 0.009 | 0.127 |
| Ac vanillic | 0.001 | 0.000 | 0.001 | 0.017 | 0.011 | 0.015 |
| Ac. Ellagic pentoside (3) | 0.014 | 0.007 | 0.011 | 0.412 | 0.274 | 0.314 |
| Benzoic acid | 0.001 | 0.001 | 0.001 | 0.017 | 0.051 | 0.025 |
| Bis galloyl HHD-glucose | 0.011 | 0.011 | 0.010 | 0.325 | 0.418 | 0.289 |
| Caffeol tartaric | 0.027 | 0.001 | 0.016 | 0.780 | 0.039 | 0.457 |
| Caffeoyl glucose (caffeic acid hexoside) | 0.002 | 0.000 | 0.001 | 0.048 | 0.000 | 0.017 |
| Caffeoyl glucose (caffeic acid hexoside) | 0.134 | 0.351 | 0.257 | 3.822 | 13.704 | 7.166 |
| Chlorogenic | 0.006 | 0.005 | 0.055 | 0.177 | 0.202 | 1.531 |
| Dimere A (Proanthocyanidins A2) | 0.009 | 0.003 | 0.005 | 0.245 | 0.105 | 0.137 |
| Dimere B (1) | 1.284 | 0.727 | 1.228 | 36.543 | 28.402 | 34.198 |
| Dimere B (2) | 0.000 | 0.000 | 0.000 | 0.012 | 0.013 | 0.012 |
| Dimere B (3) | 0.001 | 0.000 | 0.001 | 0.024 | 0.015 | 0.015 |
| Dimere B (4) | 0.001 | 0.000 | 0.001 | 0.027 | 0.004 | 0.018 |
| Dimere B (5) | 0.050 | 0.029 | 0.039 | 1.415 | 1.115 | 1.083 |
| Ellagic ac. Deoxyhexoside | 0.408 | 0.214 | 0.335 | 11.617 | 8.357 | 9.332 |
| Ellagic acid | 0.066 | 0.136 | 0.099 | 1.872 | 5.317 | 2.751 |
| Ellagitannin A (rasperberry) | 0.001 | 0.001 | 0.001 | 0.031 | 0.024 | 0.024 |
| epiafzelechin-(epi)afzelechin- | 0.027 | 0.514 | 0.247 | 0.756 | 20.094 | 6.877 |
| (epi)catechin | | | | | | |
| ferulic acid hexoside | 0.001 | 0.000 | 0.081 | 0.031 | 0.006 | 2.248 |
| Fisetin | 0.014 | 0.013 | 0.022 | 0.395 | 0.527 | 0.626 |
| Gallic acid ethyl ester | 0.000 | 0.000 | 0.000 | 0.007 | 0.011 | 0.009 |
| Gallic acid | 0.013 | 0.010 | 0.015 | 0.383 | 0.397 | 0.408 |
| galloyl-bis-HHDP-glucose | 0.056 | 0.037 | 0.055 | 1.591 | 1.449 | 1.534 |
| Galloyl-HHDP-glucose | 0.000 | 0.001 | 0.001 | 0.005 | 0.021 | 0.020 |
| Isorhamnetin | 0.001 | 0.001 | 0.001 | 0.017 | 0.028 | 0.030 |
| Kaempferol | 0.014 | 0.006 | 0.012 | 0.385 | 0.247 | 0.336 |
| Kaempferol 3-O-glucuronide | 0.810 | 0.520 | 0.689 | 23.053 | 20.314 | 19.189 |
| Kaempferol 3-O-rhamnoside | 0.000 | 0.007 | 0.003 | 0.012 | 0.292 | 0.095 |
| Kaempferol 3-O-rutinoside | 0.329 | 0.211 | 0.294 | 9.365 | 8.263 | 8.199 |
| Kaempferol-p-coumaroyl | 0.408 | 0.214 | 0.335 | 11.617 | 8.357 | 9.332 |
| m-Coumaric acid | 0.001 | 0.000 | 0.001 | 0.039 | 0.000 | 0.028 |
| Methyl-EA-Pentose | 0.000 | 0.000 | 0.000 | 0.000 | 0.000 | 0.000 |
| Methyl-EA-Pentose | 0.038 | 0.015 | 0.027 | 1.083 | 0.598 | 0.753 |
| Myricetin | 0.001 | 0.000 | 0.001 | 0.027 | 0.006 | 0.015 |
| Myricetin 3-O-arabinoside | 0.011 | 0.008 | 0.011 | 0.317 | 0.309 | 0.314 |
| Myricetin 3-O-galactoside | 0.015 | 0.011 | 0.014 | 0.429 | 0.425 | 0.381 |
| Myricetin 3-O-glucoside | 0.040 | 0.002 | 0.043 | 1.138 | 0.069 | 1.192 |
| Myricetin 3-O-rhamnoside | 0.069 | 0.045 | 0.057 | 1.955 | 1.775 | 1.602 |
| Myricetin-3-a-xylopyrannoside | 0.060 | 0.041 | 0.053 | 1.696 | 1.608 | 1.477 |
| o-Coumaric acid | 0.040 | 0.059 | 0.052 | 1.138 | 2.307 | 1.460 |
| p-Coumaric acid | 0.000 | 0.000 | 0.000 | 0.000 | 0.000 | 0.004 |
| p-Coumaroyl glucose (p-coumaroyl | 0.063 | 0.097 | 0.086 | 1.800 | 3.806 | 2.409 |
| hexose) | | | | | | |
| p-Coumaroyl glycolic acid | 0.099 | 0.127 | 0.108 | 2.824 | 4.948 | 3.019 |
| p-Coumaroyl hexose | 4.023 | 5.276 | 5.074 | 114.499 | 206.233 | 141.355 |
| p-coumaroyl sugar ester | 0.011 | 0.025 | 0.021 | 0.305 | 0.965 | 0.575 |
| p-coumaroyl-ester | 0.170 | 0.024 | 0.111 | 4.828 | 0.935 | 3.098 |
| Phloretin 2′-O-xylosyl-glucoside | 0.042 | 0.039 | 0.045 | 1.194 | 1.520 | 1.251 |
| Phloridzin | 0.070 | 0.037 | 0.057 | 1.994 | 1.439 | 1.597 |
| p-Hydroxybenzoic acid | 0.059 | 0.001 | 0.030 | 1.667 | 0.056 | 0.849 |
| p-hydroxybenzoic acid | 0.324 | 0.181 | 0.297 | 9.224 | 7.058 | 8.261 |
| Pinoresinol | 0.005 | 0.086 | 0.040 | 0.131 | 3.353 | 1.112 |
| Protocatechuic acid | 0.002 | 0.000 | 0.001 | 0.061 | 0.009 | 0.030 |
| Quercetin 3-O-galactoside | 0.001 | 0.000 | 0.001 | 0.027 | 0.009 | 0.019 |
| Quercetin 3-O-glucoside | 0.001 | 0.000 | 0.001 | 0.027 | 0.009 | 0.019 |
| Quercetin 3-O-glucuronide | 0.008 | 0.019 | 0.015 | 0.235 | 0.742 | 0.418 |
| Quercetin | 0.000 | 0.000 | 0.000 | 0.000 | 0.000 | 0.004 |
| Quercetine hexoside (arabinoside) | 0.008 | 0.004 | 0.006 | 0.235 | 0.167 | 0.170 |
| Quercetine-3-O-rutinoside | 2.484 | 0.902 | 2.326 | 70.709 | 35.258 | 64.791 |
| Resveratrol | 0.001 | 0.000 | 0.001 | 0.017 | 0.015 | 0.018 |
| Sinapic acid | 0.000 | 0.000 | 0.000 | 0.010 | 0.019 | 0.011 |
| Trimère A (1) | 0.002 | 0.001 | 0.001 | 0.048 | 0.045 | 0.031 |
| Trimère A (3) | 0.000 | 0.000 | 0.000 | 0.012 | 0.019 | 0.014 |
| Trimère A (4) | 0.001 | 0.000 | 0.001 | 0.022 | 0.011 | 0.015 |
| Trimère A (5) | 0.000 | 0.000 | 0.000 | 0.010 | 0.009 | 0.007 |
| Trimère A (6) | 0.001 | 0.000 | 0.001 | 0.027 | 0.009 | 0.014 |
| Trimère A (7) | 0.000 | 0.001 | 0.001 | 0.007 | 0.026 | 0.016 |

### Example 3—In Vitro Experiments

### Cell Culture Myocytes (L6)

A line of L6 skeletal muscle cells (kind gift of Dr Amira Klip, Hospital for Sick Children, Toronto, ON, Canada) clonally selected for high fusion potential was used in the present study. Cells were grown and maintained in monolayer culture in α-MEM medium containing 2% (vol/vol) fetal bovine serum in an atmosphere of 5% CO₂ at 37° C. Fully differentiated L6 myo-tubes were deprived of serum 5 hours before experimental procedure. Strawberry extracts (0.01×, 0.1×, 1×) were added to culture media for total treatment duration of 2 hours.

### Cell Culture Hepatocytes (FAO)

Rat hepatoma (FAO) cells were grown and maintained in monolayer culture in RPMI medium containing 10% (vol/vol) fetal bovine serum in an atmosphere of 5% CO₂ at 37° C. Cells were plated 24 hours before experiments: 4×106 cells/plate (24-well plate) for hepatic glucose production and for inflammation measurements. Fruit extracts (0.01×, 0.1×, 1×) were added to culture media for a total treatment duration of 5 hours (for glucose production) or 18 hours (for inflammation).

### Cell Culture Macrophages (J774)

Murine macrophages (J774) were grown and maintained in monolayer culture in DMEM high-glucose (25 mM) medium supplemented with 10% (vol/vol) fetal bovine serum, in an atmosphere of 5% CO₂. Cells were plated 4×10⁶/plate for 24 hours prior to the experiment in 24-well plates and treated with fruit extracts and/or LPS. Macrophages were pre-treated with fruit extracts for 16 hours. Then, cell medium was removed and LPS (100 ng/ml)+fruit or leave extracts (0.1×) containing medium was added for 6 more hours.

### Measurement of 2-Deoxyglucose Uptake

2-Dg glucose (2-deoxyglucose) uptake in L6 cells was determined in serum deprived cells stimulated in the presence or the absence of insulin (100 nM) for 30 minutes. L6 cells were rinsed once with HEPES-buffered solution (20 mM HEPES pH 7.4, 140 mM NaCl, 5 mM KCl, 2.5 mM MgSO₄, and 1 mM CaCl₂) and subsequently incubated for 8 min in transport medium (HEPES-buffered solution containing 10 μM unlabeled 2-deoxyglucose and 0.3 μCi/mL D-2-deoxy-[³H] glucose). After incubation in transport medium, cells were rinsed three times with ice-cold 0.9% NaCl solution and lysed by adding 50 mM NaOH. Cell-incorporated radioactivity was determined by scintillation counting. Protein concentrations were determined (by the BCA method) in order to normalize 2-deoxyglucose uptake and results were expressed as fold increase.

### Glucose Production Assay

After a 16 hours serum deprivation (±insulin 0.1 nM), FAO cells were washed three times with phosphate-buffered saline (PBS). Cells were then incubated for 5 h (37° C., 5% CO₂), in the presence or the absence of (0.1 nM), in a glucose production medium (glucose-free DMEM containing 2 mM sodium pyruvate, 20 mM sodium L-lactate and sodium bicarbonate [3.7 g/L]) in which fruit extracts (0.01×, 0.1×, 1×) were present. Glucose production from Fao cells was measured in the medium by a colorimetric glucose oxidase assay (Invitrogen, Burlington, Ont). Cells were lysed in 50 mM NaOH and protein content was determined (by the BCA method) in order to normalize glucose production. Results are expressed as fold change.

### Inflammation and NO Production

Nitrite accumulation in the incubation medium was used as an index of iNOS activation and NO production following inflammation. Hepatocytes (Fao) were treated for 16 hours with a cocktail of cytokines (TNF-α 10 ng/ml, IL-1β 10 000 U/ml and IFN-γ 40 ng/ml) to induce inflammation and fruit extracts at 0.1× in order to determine whether fruit extracts prevent inflammation in these cells. Macrophages (J774), were pretreated with fruit fraction for 16 hours. Then, cell medium was removed and LPS (100 ng/ml)+fruit fraction (0.1×) containing medium was added for 6 more hours. At the end of this 6 hour treatment, nitrite levels were determined spectrophotometrically using the Griess reagent: [1% (w/v) sulphanilamide/1% (w/v)N-(1-naphthyl)ethylenediamine dihydrochloride] was added to the incubation medium, and the absorption was read at 540 nm. Cells were lysed in 50 mM NaOH and protein content was determined (by the BCA method). Results are presented as % of cytokines or LPS-treated cells response, and were corrected for the protein content in the sample.

### Data Analysis

In all Figures, data represent the mean±SEM of the number of independent experiments (done in triplicate).

### Example 4—In Vitro Activity of Purified Strawberry Fractions

### Acute Effect (2 h) of Strawberry Fraction on 2-Deoxyglucose Uptake in L6 Muscle Cells.

The acute effect of strawberry extract on basal and insulin stimulated glucose transport was assessed. L6 myotubes were pre-treated for 2 hours with (0.01×, 0.1×, 1×) of strawberry fraction. Cells were then treated for 30 minutes in the presence or the absence of 100 nM insulin. No significant effect of strawberry fraction was observed on basal glucose uptake. However, strawberry fraction at 0.01× and 0.1× was found to significantly increase insulin mediated glucose uptake as compared to insulin alone (p=0.0171 and p=0.0158) respectively. As expected, all insulin treated groups were significantly different from basal (FIG. 3).

Effect of Fraction from Strawberry on Inflammation in Cytokine-Treated FAO Hepatic Cells

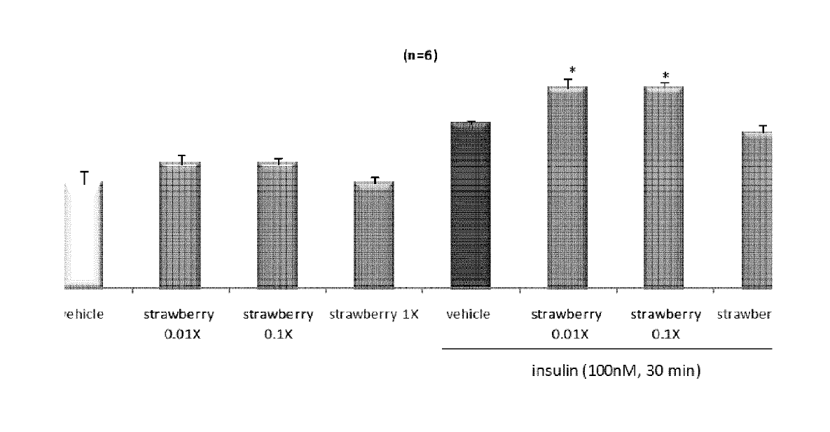

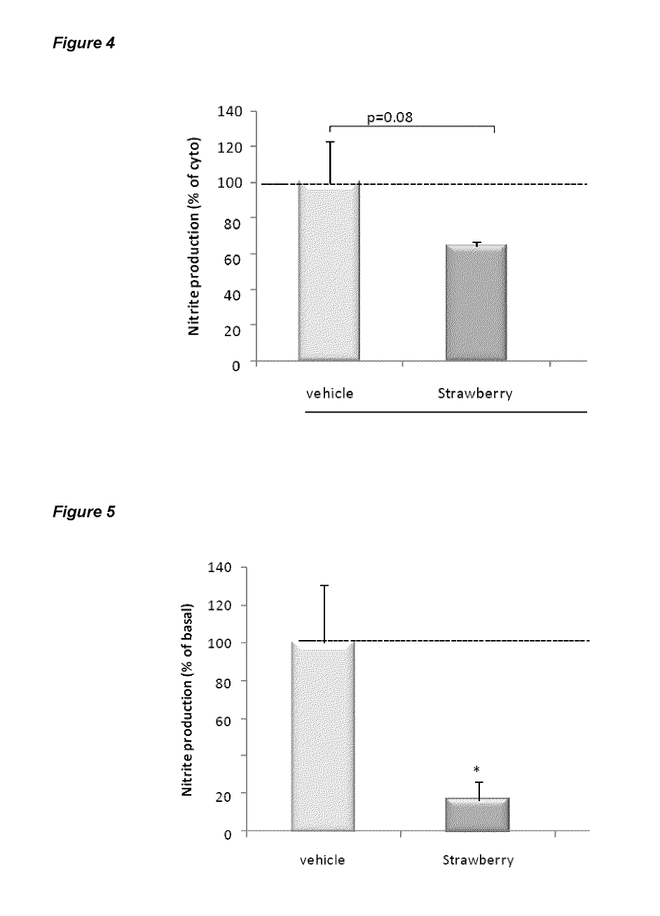

FAO hepatocytes were treated 16 hours with cytokines (TNF-α 10 ng/ml, IL-1β 10 000 U/ml and IFN-γ 40 ng/ml) in the presence or the absence of the different fruit or leaf extracts (0.1×). Nitrite production was used as an index of iNOS activity and inflammation. Strawberry tended to reduce nitrite production in cytokines treated cells (p=0.08) (FIG. 4). These experiments are preliminary (n=3).

### Effect of Strawberry Fraction on Basal Inflammation Non Treated J774 Macrophages

Macrophages (J774) were pretreated with fruit extracts (0.1×) for 22 hours. At the end of the treatment, culture medium was collected and nitrite accumulation measured as an index of iNOS activity and inflammation. Strawberry fraction was found to significantly inhibit inflammation in the basal state (p=0.02 and p=0.03) respectively (FIG. 5).

### Results and Discussion

In L6 myocytes, a 2 hour treatment with 0.01× and 0.1× of strawberry fraction was found to increase insulin stimulated glucose uptake (FIG. 3).

In LPS stimulated macrophages, no anti-inflammatory property was observed with any of the extracts (not shown). However, we found a significant inhibition of NO production with strawberry in the basal state (FIG. 5). The discrepancy between anti-inflammatory actions on basal and LPS-induced macrophages could be explained by the fact that LPS acutely and robustly induces iNOS in these cells, perhaps to a level that cannot be restrained by the extracts. However, in the basal state with relatively low inflammation, the anti-inflammatory properties of the extracts can then be revealed.

Interestingly, in cytokine stimulated FAO hepatocytes, strawberry extract also tended to reduce nitrite accumulation (FIG. 4), suggesting that the anti-inflammatory properties of this extract may help reduce liver insulin resistance and steatosis which are both linked to hepatic inflammation.

### Example 5—Plant Extracts Bioactivity

### Methods and Preliminary Results

### Preparation of Extracts

The extracts to be analyzed were in powder form. In a first series of experiments, extracts were solubilized in water, water-ethanol (80:20) mixture, ethanol or cell culture medium (RPMI). For all products, a complete dissolution was never achieved, not even with ethanol. It was therefore decided to dissolve the products in cell culture medium at a concentration of 5 mg/mL. The suspensions were then mixed by inversion (100 rpm) for 15 minutes at RT. They were then centrifuged at 16 000×g for 15 minutes at 4° C. The supernatant was collected, filtered on 0.22 μm before use. A fresh batch of extract preparation was used for each experiment.

### Activation of Cells

Before carrying out cellular assays with these extracts, toxicity was assessed on THP-1, a human monocyte cell line. Cells were grown in RPMI 10% FCS with 50 μM of 2-mercaptoethanol.

THP-1 cells (2.5×10⁵ cells/well) were transformed in macrophages by incubation with 100 nM phorbol ester (PMA) for 72 hrs. Following this PMA activation, cells were rinsed with PBS and incubated with differing concentrations of extracts (0.5, 0.1 and 0.02 μg/mL) for 24 hrs (37° C., 5% CO₂), after which the plates were centrifuged at 200 g for 10 minutes, and the supernatant collected, and frozen at −80° C. for further analysis.

### Cellular Toxicity of Extracts

Cell viability was measured at the end of incubation with the extracts. After having collected the supernatant, cells were rinsed with PBS and then incuvbated with culture medium containing 10% Alamar Blue for 4 hrs. Then, fluorescence (544ₑₓ/590ₐₘ) of reduced Alamar Blue is measured, determining the cellular activity, which is proportional to the number of live cells. Cellular viability is determined by the formula:

$$\% {viabilite}^{′} = \frac{Fluorescence {e}^{′} mise par les cellules {expose}^{′} es au produit}{Fluorescence {e}^{′} mise par les cellules incub {e}^{′} es avec milieu seul}$$

Triton (0.1%) was added in some wells as a positive control.

### Example 6—Supplement Containing Polyphenol-Rich Strawberry and Cranberry Extracts Improve Insulin Sensitivity in Insulin-Resistant, Non-Diabetic Subjects: A Parallel, Double-Blind, Placebo-Controlled and Randomized Clinical Trial

Objective:

We aimed to determine the effects of a polyphenol-rich supplement from strawberry and cranberry extracts on insulin sensitivity, glucose tolerance, insulin secretion, inflammation, oxidative stress markers and lipid profile in free-living insulin-resistant men and women with overweight or obesity.

Research Design and Methods:

In this parallel, double-blind, placebo-controlled and randomized clinical trial, 41 insulin-resistant subjects with BMI 25 completed the study. Participants in the experimental group consumed a polyphenol-rich liquid supplement (333 mg polyphenols), whereas the control group received a flavor-matched placebo supplement. All subjects were asked to take the supplement daily for 6 weeks. Insulin sensitivity (M/I) was assessed by the hyperinsulinemic-euglycemic clamp, whereas a 2-h oral glucose tolerance test (OGTT) was performed before and after the experimental period.

According to the International Diabetes Federation (2013), up to 592 million people (1 adult/10) will suffer from type 2 diabetes by the year 2035 (International Diabetes Federation, 2013). This alarming increase has been associated with several factors, including the high prevalence of obesity and sedentary lifestyles (Anderson et al, 2003; Jeon et al, 2007). In obese human individuals, elevated levels of non-esterified fatty acids, pro-inflammatory cytokines and other factors produced by adipose tissue are indeed key factors involved in the development of insulin resistance (Wellen and Hotamisligil, 2005). In insulin-resistant individuals, plasma glucose can be maintained at normal levels by compensatory increases in insulin secretion by pancreatic β-cells. Once β-cells fail to secrete the levels of insulin required to maintain normal glycemia, subjects progress to type-2 diabetes.

Over the past recent decades, scientific evidence have shown a link between increased consumption of fruits and vegetables and reduced incidence of type 2 diabetes (Mursu et al, 2014) and other chronic diseases (Boeing et al, 2012). There is also growing evidence that polyphenol consumption is associated with several beneficial effects on cardiometabolic health, particularly on glucose metabolism (Jennings et al, 2014; Hanhineva et al, 2010). According to several in vitro and animal studies, polyphenols could improve peripheral glucose uptake in insulin-sensitive tissues by increasing GLUT4 translocation and activity, reduce oxydative stress and inflammation and improve lipid metabolism (Denis et al, 2014; Breen et al, 2008; Nizamutdinova et al, 2009; Vidal et al, 2005). Berries, like strawberry, cranberry and blueberry, are known to be a particularly rich source of polyphenols (Basu and Lyons, 2012), and it has recently been demonstrated that anthocyanin-rich bilberry extract reduces glycemia and improves insulin sensitivity in diabetic mice (Takikawa et al, 2010). On the other hand, there is less documented evidence on the effects of polyphenols on glucose homeostasis and the MetS in humans (De Bock et al, 2012). So far, there have only been two studies in which the effect of berry polyphenols on insulin sensitivity was accurately assessed by the hyperinsulinemic-euglycemic clamp technique (Stull et al, 2010; Hokayem et al, 2013). According to one of them, in which obese non-diabetic insulin-resistant participants received a blueberry or a placebo smoothie twice a day, the mean percentage increase in insulin sensitivity was five times higher in the experimental group compared with the placebo group (Stull et al, 2010). In the second study using the clamp technique, the protective effect of a grape polyphenol supplement against a decrease in insulin sensitivity generated by a fructose rich diet demonstrated in overweight subjects (Hokayem et al, 2013).

To the best of our knowledge, there are no human studies on the effects of strawberry and cranberry extract rich in polyphenols on insulin sensitivity and cardiovascular risk factors in non-diabetic insulin-resistant subjects. The proposed study aims at determining the effects of a supplement rich in polyphenols from strawberry and cranberry extracts, on insulin sensitivity and related parameters in free-living men and women with overweight and insulin resistance. We hypothesized that the consumption of this supplement increases insulin sensitivity, improves lipid profile and reduces inflammatory and oxidative stress markers in overweight/obese subjects.

### Research Design and Methods

### Subjects

A total of 116 subjects, recruited in Quebec City metropolitan area by media advertising, were first screened to verify their eligibility to participate in this study. The first visit took place at the Institute of Nutrition and Functional Foods (INAF) between Spring 2012 and Fall 2013. Of the 50 eligible subjects who began the experimental period, 9 participants dropped out or were excluded during the intervention. The majority of excluded subjects (7/9) no longer met the inclusion criteria or fulfilled exclusion criteria. Two additional subjects were excluded for medical reasons from the clamp dataset. A total of 18 men and 23 post-menopausal women aged between 40 and 70 years completed the study.

All subjects were overweight or obese (BMI≧25) and insulin resistant based on fasting plasma insulin level >60 pmol·L⁻¹ (Scarsella et al, 2000) and/or the presence of impaired fasting plasma glucose (IFG) (5.6-6.9 mmol·L⁻¹) and/or impaired glucose tolerance (IGT) (7.8-11.0 mmol·L⁻¹) following a 2-h 75 g oral glucose tolerance test (Expert Committee on the Diagnosis and Classification of Diabetes Mellitus, 2003). Exclusion criteria included smoking, chronic disease (for instance, diabetes), metabolic or acute disease, use of medication known to affect lipid or glucose metabolism, major surgery in the 3 months preceding the study, significant weight change (±10%) within 6 months prior to beginning the study and having an allergy or an intolerance to strawberry and/or cranberry. This study was approved by the Research Ethical Committee of The Quebec University Health Center. Informed written consent was obtained from all the participants after reading a detailed consent form prior to their participation to the study.

Experimental Design.

This 6-week parallel-arm study was double-blinded, placebo-controlled, and randomised. Participants were equally divided in 2 groups after a 2-week run-in period. Participants in the treatment group consumed a polyphenol-rich supplement, whereas the control group received a matched placebo. All subjects were asked to consume the supplement daily for a 6-week period. During both run-in and experimental periods, subjects were asked to maintain their usual food habits and physical activity level and were limited to one unit drink or less of beer or spirits per day. The consumption of berries, wine, polyphenol supplements and all products containing berries or wine was also forbidden throughout the entire study period. During the experimental period, a registered dietitian called all participants to ensure compliance and progress of the project. To document compliance, subjects were requested to bring back the unused bottles at the end of the study. Bottle counts indicated that 99% of the supplements in both groups were taken. Also, a 6-week checklist was provided to all participants to identify supplements or placebo that had not been ingested.

Supplements.

The supplement and placebo were isoenergetic, and had the same visual aspect and taste. Both supplement and placebo were formulated by Atrium Innovations Inc. (Quebec, Canada) and were provided as liquid preparations (120 ml per day). The experimental supplement contained 1.84 g of a blend of strawberry and cranberry extracts (GlucoPhenol™) and provided a daily dose of 333 mg of polyphenols. The strawberry-cranberry extracts blend, supplied by Nutra Canada Company (Quebec, Canada), was characterized for its phenolic composition as previously described (Dudonné et al, 2014). This dose corresponds approximately to the amount of polyphenols provided by 250 g of fresh strawberries and cranberries.

Anthropometric and blood pressure measurements. Body weight, height, waist and hip circumferences were measured at the beginning and at the end of the study. The waist circumference was measured three times at the mid-distance between the iliac crest and the last rib margin with a flexible inextensible plastic tape to the nearest millimeter. Hip circumference was also measured three times at the largest point below the waist with the same flexible inextensible plastic tape. BMI and the waist-to-hip ratio were then calculated. Blood pressure was measured 3 times on the right arm with an automatic tensiometer following a 10-minute rest at the beginning and the end of the experimental period.

Food records and questionnaires. During the screening visit, 2 online self-administered questionnaires were completed by all subjects to collect information on medical history, lifestyle, economic and socio-demographic characteristics. Participants were also asked to complete 2 online self-administered questionnaires at the beginning and at the end of the experimental period, including a validated food frequency questionnaire (FFQ) (Labonté et al, 2012), and a short physical activity questionnaire. There was also an additional questionnaire on subject satisfaction and side effects related to the liquid supplement at the end of the study. Changes in medication, temporary medication, natural health products intake or consumption of any other food supplements were monitored during the entire study period.

Oral Glucose Tolerance Test (OGTT).

A 75-g oral glucose tolerance test was performed before and after the experimental period at the Institute of Nutrition and Functional Foods to assess glucose tolerance after a 12-h overnight fast. Alcohol intake was forbidden 48 h before the test. For the second OGTT, participants were asked to consume the liquid supplement 12 h before their OGTT appointment. Blood samples were taken at timepoint −15, 0, 15, 30, 60 and 120 min kept at −20° C. for measurement of glucose, insulin and C-peptide concentrations.

Hyperinsulinemic-Euglycemic Clamp.

A 120-min hyperinsulinemic-euglycemic clamp was performed once before and after the experimental period at the Diabetes Research Unit of the Laval University Health Center after a 12-h overnight fast according to the method described in Piché et al. (2005). This test is considered as the gold standard for assessing insulin sensitivity (DeFronzo et al, 1979). Alcohol intake was forbidden 48 h before the clamps. Here again, participants were asked to consume the liquid supplement 12 h before their appointment for the second clamp. The insulin-stimulated glucose disposal rate (GDR or M) was established from glucose infusion rate (mg·min⁻¹) divided by body weight (kg) during the final 30 min of the clamp. The insulin sensitivity index (M/I) was calculated from the M value divided by the mean insulin concentration during the final 30 minutes of the clamp (mg·kg⁻¹·min⁻¹·pmol⁻¹) (DeFronzo et al, 1979).

Biochemical Analyses.

Plasma samples were collected in the fasting state before each OGTT/clamp, immediately centrifuged and stored at −20° C. for further analysis of plasma lipids, inflammatory markers (high-sensitivity C-reactive protein (hsCRP), Interleukin-6 (IL-6), Tumor Necrosis Factor-alpha (TNF-α), High molecular weight (HMVV) adiponectin and Regulated on Activation, Normal T Cell Expressed and Secreted (RANTES)/CCL5), plasminogen activator inhibitor-1 (PAI-1), a marker of cardiovascular risk, ferric reducing antioxidant power (FRAP) and oxidized LDL, markers of oxidative stress. During the clamp, additional blood samples (2 ml) were collected at 0, 30, 60, 90 and 120 min to measure serum free fatty acids (FFA) concentrations. Serum samples for FFA measurements were centrifuged after 30 min at room temperature and then stored at −80° C. until analysis.

Glucose, Insulin, C-Peptide.

Plasma glucose was determined using an enzymatic method (Desbuquois and Aurbach, 1971) and plasma insulin was measured by radioimmunoassay with polyethylene glycol separation (Richterich and Dauwalder, 1971). Plasma C-peptide level, an indicator of insulin secretion used to estimate pancreatic β-cell function, was determined using a modified version of the method of Heding with polyclonal antibody A-4741 from Ventrex (Portland, Me.) and polyethylene glycol precipitation (Desbuquois and Aurbach, 1971).

Lipids and Lipoproteins.

Plasma LDL and HDL were isolated from fresh blood by ultracentrifugation combined with a heparin-manganese chloride precipitation (Burstein and Samaille, 1960; Moorjani et al, 1986). Then cholesterol and triglyceride concentrations in total plasma and lipoproteins were determined enzymatically by using a Technicon RA-500 analyzer (Bayer, Tarrytown, N.Y.) (Moorjani et al, 1986). Blood samples were kept at −20° C. until analysis. FFA were determined via an enzymatic colorimetric assay (Wako Diagnostics, Richmond, USA) by using a Beckman Olumpus AU400.

Inflammatory, Cardiovascular and Oxidative Stress Markers.

Serum level of hs-CRP was measured using nephelometry as described previously (Piché et al, 2005). PAI-1, IL-6 and TNF-α were measured in plasma, at the Quebec Heart and Lung Institute, Quebec, using commercially available Multiplex methods. Cytokines (IL-6 and TNF-α) and PAI-1 concentrations were quantified by a Milliplex kit (EMD Millipore, USA). Plates were read and analyzed using the Bioplex 200 system (BioRad, USA). Oxidized LDL, HMW adiponectin and RANTES were determined using a commercially available enzyme-linked immunosorbent assay (ELISA) (Mercodia, Sweden; RnDSystems, USA) according to manufacturer's instructions. Total antioxidant capacity of plasma, assessed by FRAP assay, was determined as described previously (Rubio et al, 2014).

Statistical Analyses.

Power calculation at 80% from data published by Stull et al. (2010) and ours showed that a minimum of 40 subjects, 20 in each group, was required to observe significant changes in insulin sensitivity over a 6-week dietary intervention, taking into account 25% expected dropouts. Statistical analyses were performed using SAS 9.3 (SAS Institute, Cary N.C.). PROC MIXED for ANCOVA with baseline insulin sensitivity as covariate, was used to compare the changes in M/I and GDR with the 2 treatments. A two-way repeated-measures ANOVA was applied for variables with repeated measures over time (glucose, insulin, C-peptide and FFA concentrations during the OGTT or clamp). Furthermore, positive incremental area under the curve (IAUC) for glucose (mmol·L⁻¹·min⁻¹), insulin (pmol·L⁻¹·min⁻¹) and C-peptide (pmol·L⁻¹·min⁻¹) were calculated using the trapezoid method with baseline value corresponding to the fasting level (timepoint−15 min of the OGTT). PROC MIXED for a two-way ANOVA was used to compare the changes on positive IAUC for variables measured during the OGTT (glucose, insulin and C-peptide), on anthropometric and blood pressure measurements, FFQ variables, lipid and cardiovascular parameters as well as markers of inflammation and oxidative stress prior and after the 2 treatments. Correlation coefficients were calculated using Pearson's method in order to detect associations between variables. A statistically significant level of P≦0.05 was applied for all tests and the results presented are means±standard errors of the mean (SEM).

### Results

Results:

The polyphenol-rich supplement significantly (P=0.03) increased insulin sensitivity (+0.9±0.5) as compared to the placebo (−0.5±0.5) in overweight and obese subjects. Compared to the polyphenol-rich supplement group, participants in the placebo group had a significantly higher first phase insulin secretion response as measured by C-peptide levels during the first 30 minutes of the OGTT (P=0.002). No significant differences were observed for inflammatory and oxidative stress markers, nor for the lipid measurements.

Subject Baseline Characteristics.

Baseline clinical and laboratory characteristics of all participants are shown in Table 3. All subjects were insulin resistant, overweight or obese (BMI≧25 kg·m⁻²) with increased abdominal adiposity (waist circumference >94 cm for men and >80 cm for women). There were no significant differences between the 2 groups regarding age, body weight, BMI, waist and hip circumferences, plasma lipids, fasting plasma glucose, 2-h plasma glucose or fasting plasma insulin.

TABLE 3
Baseline characteristics of subjects

| | All | Supplement | Placebo | |
|---|---|---|---|---|
| | (n = 41) | (n = 20) | (n = 21) | P value* |
| Men/women (n/n) | 17/23 | 8/11 | 9/12 | — |
| Age (years) | 58 ± 1 | 57 ± 1 | 60 ± 1 | 0.18 |
| Body weight (kg) | 85 ± 2 | 85 ± 3 | 85 ± 3 | 0.97 |
| BMI (kg · m⁻²) | 31 ± 1 | 31 ± 1 | 31 ± 1 | 0.91 |
| Waist circumference (cm) | 104 ± 2 | 104 ± 3 | 104 ± 2 | 0.95 |
| Hip circumference (cm) | 111 ± 1 | 111 ± 2 | 111 ± 2 | 0.93 |
| Cholesterol (mmol · L⁻¹) | | | | |
| Total | 5.53 ± 0.14 | 5.70 ± 0.17 | 5.37 ± 0.22 | 0.07 |
| HDL | 1.29 ± 0.04 | 1.25 ± 0.05 | 1.33 ± 0.05 | 0.24 |
| LDL | 3.36 ± 0.12 | 3.52 ± 0.17 | 3.20 ± 0.15 | 0.18 |
| Total TG (mmol · L⁻¹) | 1.88 ± 0.17 | 2.03 ± 0.24 | 1.73 ± 0.26 | 0.39 |
| Total chol./HDL | 4.4 ± 0.2 | 4.8 ± 0.3 | 4.1 ± 0.2 | 0.07 |
| chol. ratio | | | | |
| Fasting plasma | 5.9 ± 0.1 | 6.0 ± 0.1 | 5.8 ± 0.1 | 0.16 |
| glucose (mmol · L⁻¹) | | | | |
| 2-h plasma glucose | 7.5 ± 0.3 | 7.7 ± 0.4 | 7.4 ± 0.4 | 0.71 |
| (mmol · L⁻¹) | | | | |
| Fasting plasma | 124 ± 8 | 118 ± 11 | 130 ± 11 | 0.45 |
| insulin (pmol · L⁻¹) | | | | |

Values are means ± standard errors of the mean (SEM).
*P values assessed by PROC MIXED ANOVA between the two groups. TG, triglycerides.

At baseline, all subjects had a high fasting plasma insulin level (>60 pmol·L⁻¹), of which 31 subjects had fasting plasma insulin levels >90 pmol·L⁻¹. From data collected during the pre-intervention OGTT and according to the Expert Committee on the Diagnosis and Classification of Diabetes Mellitus (2003), 12 subjects had both IFG (5.6-6.9 mmol·L⁻¹) and IGT (7.8-11.0 mmol·L⁻¹), 17 subjects had IFG only, 3 subjects had IGT only and 9 among them had normal glucose tolerance (NGT) (Fasting plasma glucose <5.6 mmol·L⁻¹ and plasma glucose <7.8 mmol·L⁻¹ after 120 minutes).

Food Consumption.

According to FFQ data, there were no differences in baseline food consumption between the groups. Furthermore, no significant differences in energy and macronutrient intake (Post-Pre) were detected between the 2 groups.

Anthropometric Measurements and Blood Pressure.

Body weight, anthropometric, systolic and diastolic blood pressure measurements were performed at the beginning and the end of the experimental period. No significant changes were observed for these parameters between the two groups (not shown).

Insulin Sensitivity and Other Parameters of Glucose Homeostasis.

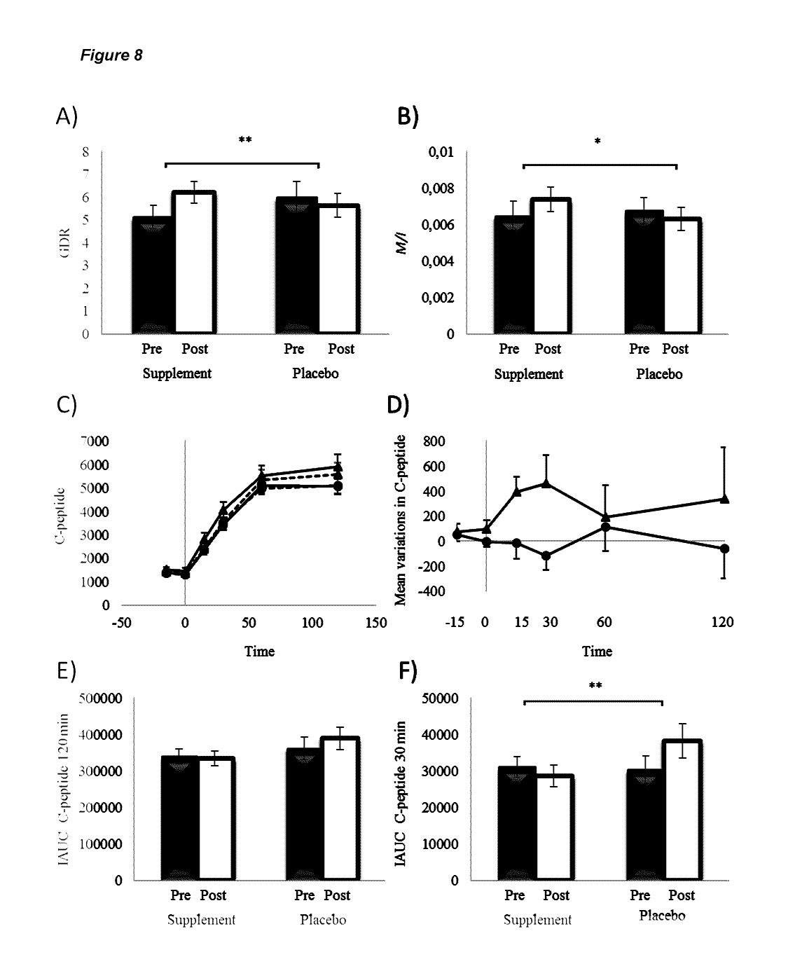

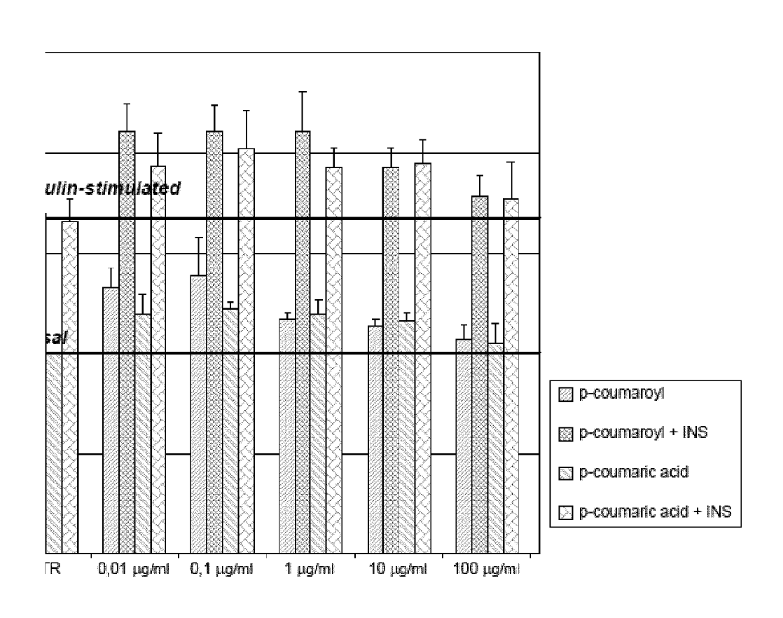

The polyphenol-rich supplement increased the glucose disposal rate (M) by 21% (FIG. 8A) and insulin sensitivity (M/I) by 14% (FIG. 8B). In contrast to the placebo control group, glucose disposal rate (M) decrease by 6% and insulin sensitivity (M/I) by 7% in the polyphenol group. When variations due to the 2 treatments were compared to each other, there was a significant improvement in glucose disposal rate (P=0.007) and insulin sensitivity (P=0.03) with the polyphenol-rich supplement compared with the placebo. The mean change in MIl was significantly increased in the polyphenol group (+0.9±0.5×10³ mg·kg⁻¹·min) compared to the placebo group (−0.5±0.5×10³ mg·kg⁻¹˜min⁻¹·pmol⁻¹) (P=0.03). Similarly, an improvement in the GDR (M) was observed in the polyphenol group versus the placebo, with a mean change of +1.1±0.4 mg·kg⁻¹·min⁻¹ and −0.4±0.4 (P=0.007) respectively.

We also performed repeated measurements ANOVA for glucose (Table 4), insulin (Table 4) and C-peptide (Table 4, FIGS. 8C and 8D) over time during OGTT and for FFA (not shown) over time during clamp. There were no differences in baseline values between groups for all glucose metabolism parameters and for FFA. However there was an overall increase in plasma C-peptide with the placebo compared with the polyphenol-rich supplement (P=0.002). No differences between treatments were observed for plasma glucose (P=0.31), plasma insulin (P=0.21) and serum FFA (P=0.95) between the treatments.

TABLE 4
IAUC and timepoint values over time during OGTT for glucose, insulin and
C-peptide before and after the experimental period.

| | Supplement (n = 20) | | Placebo (n = 21) | | |
|---|---|---|---|---|---|
| | Pre | Post | Pre | Post | P value |
| Glucose (mmol · L⁻¹) | | | | | |
| −15 | 6.1 ± 0.1 | 6.1 ± 0.1 | 5.9 ± 0.1 | 6.0 ± 0.1 | 0.31* |
| 0 | 6.0 ± 0.1 | 6.1 ± 0.1 | 5.8 ± 0.1 | 5.9 ± 0.1 | |
| 15 | 8.1 ± 0.2 | 8.1 ± 0.3 | 7.7 ± 0.2 | 8.0 ± 0.2 | |
| 30 | 9.7 ± 0.3 | 9.8 ± 0.3 | 9.3 ± 0.3 | 9.4 ± 0.3 | |
| 60 | 10.3 ± 0.5 | 10.6 ± 0.4 | 9.9 ± 0.5 | 9.5 ± 0.4 | |
| 120 | 7.7 ± 0.4 | 7.5 ± 0.4 | 7.4 ± 0.4 | 6.9 ± 0.3 | |
| IAUC glucose up to | 348 ± 31 | 357 ± 32 | 329 ± 34 | 291 ± 27 | 0.16^{†} |
| 120 min | | | | | |
| IAUC glucose 30 | 56 ± 4 | 58 ± 6 | 55 ± 5 | 3 ± 4 | 0.77^{†} |
| up to min | | | | | |
| Insulin (pmol · L⁻¹) | | | | | |
| −15 | 129 ± 11 | 132 ± 14 | 134 ± 12 | 144 ± 16 | 0.21* |
| 0 | 118 ± 9 | 120 ± 13 | 130 ± 13 | 131 ± 17 | |
| 15 | 402 ± 37 | 418 ± 53 | 479 ± 58 | 600 ± 85 | |
| 30 | 729 ± 73 | 645 ± 76 | 759 ± 79 | 822 ± 106 | |
| 60 | 1006 ± 99 | 968 ± 104 | 1173 ± 148 | 1064 ± 127 | |
| 120 | 895 ± 111 | 818 ± 99 | 1094 ± 184 | 1208 ± 233 | |
| IAUC insulin up to | 80 ± 8 | 74 ± 8 | 95 ± 12 | 95 ± 13 | 0.51^{†} |
| 120 min | | | | | |
| IAUC insulin up to | 9 ± 1 | 8 ± 1 | 10 ± 1 | 12 ± 2 | 0.13^{†} |
| 30 min | | | | | |
| C-peptide (pmol · L⁻¹) | | | | | |
| −15 | 1347 ± 86 | 1397 ± 111 | 1433 ± 95 | 1506 ± 122 | 0.002* |
| 0 | 1301 ± 83 | 1299 ± 106 | 1357 ± 101 | 1456 ± 126 | (FIG. 8C |
| | | | | | & 8D) |
| 15 | 2354 ± 151 | 2337 ± 182 | 2416 ± 221 | 2807 ± 280 | |
| 30 | 3528 ± 198 | 3413 ± 224 | 3583 ± 296 | 4045 ± 359 | |
| 60 | 4980 ± 243 | 5090 ± 316 | 5338 ± 442 | 5529 ± 424 | |
| 120 | 5113 ± 348 | 5055 ± 337 | 5576 ± 481 | 5914 ± 524 | 0.19^{†} |
| IAUC C-peptide up | 340 ± 20 | 335 ± 20 | 363 ± 30 | 390 ± 31 | (FIG. 8F) |
| to 120 min | | | | | 0.003^{†} |
| IAUC C-peptide up | 31 ± 3 | 29 ± 3 | 30 ± 4 | 38 ± 5 | (FIG. 8E) |
| to 30 min | | | | | |

Values are means ± standard errors of the mean (SEM).
*P values assessed by repeated measures ANOVA between the variations of the two groups.
^{†}P values assessed by PROC MIXED ANOVA between the variations of the two groups. IAUC, positive incremental area under the curve. IAUC glucose (mmol · L⁻¹ · min⁻¹), IAUC insulin (× 10³ pmol · L ⁻¹ · min⁻¹), IAUC C-peptide (× 10³ pmol · L⁻¹ · min⁻¹).

The mean IAUC up to 30 min and 120 min after the OGTT for plasma glucose, insulin and C-peptide are shown in Table 4. No significant differences in the IAUC for plasma glucose (P=0.16) (Table 4), insulin (P=0.51) (Table 4) and C-peptide (P=0.13) (Table 4, FIG. 8E) were observed up to 120 min. However the IAUC up to 30 min for plasma C-peptide was reduced by 8% after polyphenol consumption and increased by 26% after placebo, leading to a significant difference between the two groups (P=0.003) (Table 4, FIG. 8F).

Plasma Lipid Concentrations.

No differences in total cholesterol and triglycerides (TG), LDL and HDL cholesterol were observed between the two groups (Table 5).

TABLE 5
Plasma lipids and inflammatory, cardiovascular and oxidative stress
markers and total antioxidant capacity before and after the experimental period.

| | Polyphenol (n = 20) | | Placebo (n = 21) | | |
|---|---|---|---|---|---|
| | Pre | Post | Pre | Post | P value* |
| Cholesterol (mmol · L⁻¹) | | | | | |
| Total | 5.70 ± 0.17 | 5.60 ± 0.19 | 5.37 ± 0.22 | 5.45 ± 0.20 | 0.33 |
| HDL | 1.25 ± 0.05 | 1.26 ± 0.06 | 1.33 ± 0.05 | 1.37 ± 0.06 | 0.40 |
| LDL | 3.52 ± 0.17 | 3.51 ± 0.17 | 3.20 ± 0.15 | 3.37 ± 0.17 | 0.32 |
| Total chol./HDL chol. | 4.76 ± 0.27 | 4.62 ± 0.24 | 4.12 ± 0.20 | 4.08 ± 0.17 | 0.41 |
| ratio | | | | | |
| TG (mmol · L⁻¹) | 2.03 ± 0.24 | 1.82 ± 0.21 | 1.73 ± 0.26 | 1.56 ± 0.18 | 0.99 |
| CRP (mg · L⁻¹) ^{†} | 3.6 ± 0.7 | 3.0 ± 0.6 | 5.4 ± 2.9 | 3.0 ± 0.6 | 0.53 |
| TNF-α (ng · L⁻¹) ^{‡} | 4.4 ± 0.4 | 4.0 ± 0.4 | 4.3 ± 0.2 | 4.0 ± 0.3 | 0.69 |
| IL-6 (ng · L⁻¹) ^{‡} | 4.9 ± 0.4 | 4.8 ± 0.6 | 5.6 ± 1.0 | 4.9 ± 0.8 | 0.23 |
| HMW adiponectin | 6830 ± 1094 | 5913 ± 934 | 7147 ± 1145 | 6725 ± 1199 | 0.65 |
| (ng · mL⁻¹) ^{§} | | | | | |
| PAI-1 × 10³ (ng · L⁻¹) ^{‡} | 30.5 ± 2.7 | 28.5 ± 3.1 | 30.0 ± 2.5 | 27.3 ± 3.5 | 0.87 |
| Oxidized-LDL (U · L⁻¹) ^{†} | 96.5 ± 6.2 | 92.9 ± 5.6 | 79.7 ± 5.4 | 80.2 ± 4.8 | 0.22 |
| FRAP (μMFe2+) ^{†} | 1191 ± 58 | 1237 ± 65 | 1135 ± 36 | 1189 ± 41 | 0.70 |
| RANTES (ng · L⁻¹) ^{†} | 3214 ± 392 | 3146 ± 438 | 3063 ± 401 | 2768 ± 369 | 0.71 |

Values are means ± standard errors of the mean (SEM).
*P values assessed by PROC MIXED ANOVA between the variations of the two groups.
^{†} n = 39.
^{‡} n = 38.
^{§} n = 33.
TG, triglycerides; hsCRP, high-sensitivity C-reactive protein;
TNF-α, Tumor Necrosis Factor-alpha;
IL-6, Interleukin-6;
HMW, High molecular weight;
PAI-1, plasmiogen activator inhibitor-1;
FRAP, ferric reducing antioxidant power;
RANTES, Regulated on Activation, Normal T Cell Expressed and Secreted.

Plasma Inflammatory, Oxydative Stress and CVD Markers.

The effects of the polyphenol-rich supplement on inflammatory and oxydative stress markers are shown in Table 5. No significant differences in pro-inflammatory cytokines, hsCRP, HMW adiponectin, PAI-1, oxidized-LDL, RANTES and total antioxidant capacity of plasma (FRAP) were observed.

Sex Effect.

There was no sex effect nor sex by treatment interaction for any variable.

### Conclusion

Conclusion:

Dietary supplementation with a polyphenol-rich supplement from strawberry and cranberry extracts improved insulin sensitivity in overweight and obese insulin-resistant men and women.

This study investigated the effect of daily consumption of a polyphenol-rich supplement from strawberries and cranberries in insulin-resistant subjects for a period of 6 weeks. The main findings are the following: 1) an improvement in insulin sensitivity, as assessed by the hyperinsulinemic-euglycemic clamp, and 2) prevention of further early compensatory insulin secretion, as shown by a lack of increase in the early C-peptide response during an OGTT.

This study demonstrated a significant improvement of insulin sensitivity and glucose disposal rate following the consumption of the combination of strawberry and cranberry extracts rich in polyphenols compared with the placebo. These results are in good agreement with those of Stull et al. (2010) who observed a 22% increase in insulin sensitivity following daily dietary supplementation with whole blueberries in obese, non-diabetic, and insulin-resistant human subjects, and those of Hokayem et al (2013) who noted that the negative effects of fructose used to promote insulin resistance were counteracted by grape polyphenol supplementation in a double-blind controlled trial. It is noteworthy that we used a much smaller dose of polyphenols (a total of 333 mg of polyphenols from combined strawberry and cranberry extracts/day) as compared to the one used by Stull et al. (2010) (1462 mg from blueberry extract/day) and Hoyakem et al. (2013) (2 g from grape polyphenols/day). Therefore the present results further suggest that polyphenols are even more potent to reduce insulin resistance than previously thought and/or that specific polyphenols from strawberries and cranberries may have a greater impact than those of other fruits to reduce insulin resistance and risk of type 2 diabetes.

It should be mentioned that other studies failed to observe an effect of berries (Kar et al, 2009; Basu et al, 2010; Lee et al, 2008) or anthocyanin supplement (Hassellund et al, 2013) on insulin sensitivity in humans. Differences in the techniques used to assess insulin sensitivity between these studies may explain these discrepancies. In a similar way to the present report, Stull et al. (2010) and Hokayem et al (2013) assessed insulin sensitivity by using the hyperinsulinemic-euglycemic clamp methods, recognized as the gold standard method for measurement of whole-body insulin sensitivity. Conversely, other studies (Kar et al, 2009; Basu et al, 2010; Lee et al, 2008; Hassellund et al, 2013) calculated the homeostasis model assessment of insulin resistance (HOMA-IR), a fasting index related to a greater extent to hepatic insulin resistance.

The progression from NGT to type 2 diabetes is characterized by both an increase in insulin resistance and a decrease in insulin secretion caused by β-cell dysfunction. Insulin resistance is defined as decreased tissue sensitivity to insulin to stimulate glucose uptake and utilization. In the early stages of insulin resistance, plasma glucose is maintained at normal levels by a compensatory increase in insulin secretion, the first abnormality being an increase in first-phase insulin secretion by pancreatic β-cells (Kahn et al, 1993). But when β-cell compensation fails, fasting plasma glucose levels rise (IFG), leading to impaired glucose tolerance (IGT) and eventually type 2 diabetes (Pratley and Weyer, 2002). In the context of the present study, the liquid supplement rich in polyphenols prevented a further elevation in early-phase insulin release, as indicated by C-peptide levels, and in the overall increase of insulin secretion, suggesting that the improvement in insulin sensitivity after consumption of the supplement rich in polyphenols may have precluded a further compensatory increase in insulin secretion.

The beneficial effects of this supplement cannot be explained by variations in energy and macronutrient intake, body weight, body fat mass, plasma inflammatory, cardiovascular and oxidative stress markers since no changes in these parameters were observed between the 2 groups. Moreover, all subjects fully complied with the instructions not to eat berries or drink wine during the experimental period, according to food intake data. According to the USDA (36) and Phenol-Explorer (37) databases and data from Brat et al (2006), the difference in the consumption of polyphenols from wine and berries from day 0 to the end of the 6-week experimental period in both groups was negligible (−27±23 mg for the polyphenol group and −5±4 mg for the placebo group). Therefore, dietary intake of polyphenols was unlikely to be a confounder in the interpretation of our results. Further, dietary intake data calculated from from FFQ administered prior to the 2 wk run-in period (i.e. prior to the restriction of wine and berries) indicated a typical polyphenol intake in our population of approximately 200 mg/d (data not shown). Thus, the intervention providing 300 mg of polyphenols per day achieved an incremental increase in polyphenol intake of 100 mg per day over the typical diet in this population. While the current study demonstrated the specific benefits of supplementing with 300 mg of a particular polyphenol blend from strawberries and cranberries, it is of interest to consider whether supplementing with 100 mg per day in addition to a healthy diet rich in berries and moderate wine consumption could yield similar benefits.

Anthocyanins, proanthocyanidins, ellagitanins, phenolic acids and quercetins were the most abundant polyphenols in the strawberry-cranberry extracts blend (Dudonné et al, 2014). These polyphenols thus ameliorate insulin sensitivity most likely by improving insulin signaling and increasing glucose transport in skeletal muscle cells. In this respect, Nizamutdinova et al (2009) showed that anthocyanins administration by gavage can improve insulin signaling by stimulating tyrosine phosphorylation of the insulin receptors, and by increasing expression of GLUT4 glucose transporters in muscle of STZ-diabetic rats. Similarly, Anhê et al (2012) demonstrated that quercetin can upregulate the GLUT4 expression in muscle cells and thus improve insulin sensitivity in diabetic mice.

Previous in vitro studies have shown a beneficial impact of p-coumaric acid on AMPK, a key energy-sensing pathway, leading to increased glucose uptake in muscle cells (Bhattacharya et al, 2013; Yoon et al, 2013). Another potential mechanism underlying the beneficial effects of the polyphenol supplement on insulin sensitivity is the modulation of the gut microbiota. Indeed, we have recently shown that cranberry polyphenols can improve insulin sensitivity in high-fat fed mice through modulation of the gut microbiota, leading to reduced inflammation in both intestinal and hepatic tissues (Ahnê et al, 2014).

The participants of this study were insulin resistant and included both genders and a relatively broad age range (40-70 y). Given the free-living nature of the study, the results presented in the present study could be, to some extent, generalizable to an adult prediabetic population in Western countries. However we did not determine and correlate directly strawberry and cranberry polyphenols and their metabolites in plasma or urine with insulin sensitivity outcomes and related parameters. Furthermore, muscle and adipose tissue biopsies would have allowed to verify if the polyphenol-rich supplement consumption reduced inflammation in these tissues. These would also have permitted the uncovering of the molecules involved in cellular insulin signaling. Nonetheless, considering the robust nature of our randomized, placebo-controlled, double-blind, parallel-arm design, it is most than likely that our study outcomes resulted from the consumption of polyphenol-rich supplement from strawberry and cranberry. Because our study was short term and had a relatively small number of subjects, larger and longer-term trials are still required to confirm and expand upon the potential role of strawberry and cranberry extracts in preventing or delaying the onset of type 2 diabetes.

In conclusion, these data indicate that consumption of this combination of strawberry and cranberry extracts rich in polyphenols may improve insulin sensitivity and prevent an increase in compensatory insulin secretion, and could therefore represent a promising alternative approach to improve glucose homeostasis in subjects at risk of type 2 diabetes. Further controlled dose-response trials are needed to determine the optimal dose of this polyphenol-rich supplement to use in future larger and longer-term studies.

### Example 7—Concentration of Coumaric Acid and Derivatives in Authentique Orléans Strawberry

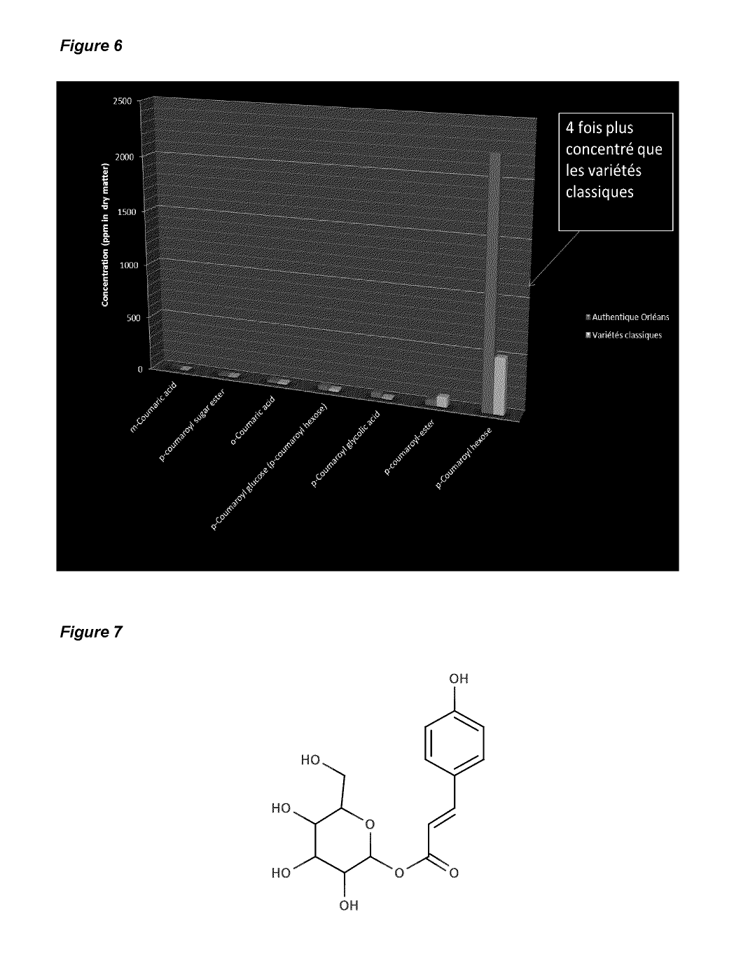

FIG. 6 shows that the present purification process when used in conjunction with pulp from the strawberry variety Authentique Orléans yields an extract that is highly enriched in a particular derivative of coumaric acid such as p-coumaroyl hexose, the structure of which is shown in FIG. 7.

Particularly, Table 6 demonstrates that different strawberry varieties possess dramatically different concentrations in p-coumaroyl hexose and that the Authentique Orléans variety, even as raw fruit paste, can achieve unexpectedly high concentrations of this bioactive molecule.

Surprisingly, the Authentique Orléans variety yields a raw fruit paste that contains at least 1000 ppm in p-coumaroyl hexose. In addition, when the fruit paste is submitted to the extraction process as defined herein, extracts are obtained that can achieve at least about 4000 ppm of p-coumaroyl hexose of dried matter, particularly at least about 5000 ppm, more particularly at least about 6000 ppm.

TABLE 6
Concentrations in p-coumaroyl hexose of various products from
strawberry and raspberry processing.

| | p-coumaroyl- |
|---|---|
| | hexose (ppm) |
| Origin of paste/extract | from MS |
| Aut. d'Orléans (strawberry fruit paste) | 1131.9 |
| Albiom champ (strawberry fruit paste) | 120.3 |
| Albiom serre (strawberry fruit paste) | 92.1 |
| Monterey champ (strawberry fruit paste) | 299.6 |
| Monterey tunel (strawberry fruit paste) | 85.9 |
| SeaScape champ (strawberry fruit paste) | 163.9 |
| St-Jean champ (strawberry fruit paste) | 390.5 |
| Sweet charlie (strawberry fruit paste) | 59.5 |
| Glucophenols | 4374.7 |
| Nutra 3464 | 6254.0 |
| Nutra 3466 (strawberry extract advantage 2% | 948.1 |
| TP) | |
| Nutra 3467 (strawberry extract select 10% | 5457.7 |
| TP) | |
| Nutra 3473 (strawberry leaves) | 1156.0 |
| Nutra 3474 (raspberry leaves) | 0.00 |
| Strawberry (fresh green fruits) | 9 |
| Strawberry Albion (lyophilised leaves) | 0.0 |
| Strawberry Authantique d'Orléans (lyophilised | 0.0 |
| leaves) | |
| Strawberry Monterry (lyophilised leaves) | 0.0 |
| Sea buckthorn Leikora (lyophilised leaves) | 0.0 |
| Sea buckthorn Russian Orange (lyophilised | 0.0 |
| leaves) | |
| Sea buckthorn Tatjana (lyophilised leaves) | 0.0 |
| Raspberry A. brittem (lyophilised leaves) | 0.0 |
| Raspberry Polka (lyophilised leaves) | 0.0 |
| Raspberry Jeanne d'Orléans (lyophilised leaves | 0.0 |

On the other hand, other anti-oxidant fruits that possess high concentrations of polyphenols, such as raspberry and sea buckthorn, are shown not to possess any levels of p-coumaroyl hexose, demonstrating that the two types of bioactive molecules do not go hand in hand.

### Example 8—Effects of p-Coumaroyl Hexose and p-Coumaric Acid on Basal and Insulin-Stimulated Glucose Uptake

The objective of these experiments was to determine the bioactivity of the p-coumaric acid which is the molecule found in the circulation after intestinal absorption of the strawberry p-coumaroyl hexose molecule. These experiments are representing 4 independent experiments performed in triplicates.

2-Dg glucose uptake measurements were performed in L6 muscle cells to evaluate and compare the capacity of p-coumaroyl and p-coumaric acid to stimulate basal and insulin-mediated glucose transport.

In the basal situation, in absence of insulin, as compared to control situation (FIG. 9, CTL first and third bars), we observed that both molecules were able to induce basal glucose transport, especially at the lower concentration. In the insulin-stimulated condition, as compared to the control situation (FIG. 9, CTL second and fourth bars), we observed that both molecules increased insulin-stimulated glucose transport, again, at the lower concentration.

### CONCLUSION

The original aim of the present invention was to provide a method for the extraction of berry polyphenols. Surprisingly, following the extraction of different strawberry varieties, high concentrations of p-coumaroyl hexose were found when carried out in the Authentique Orléans variety.

Even more surprisingly, this derivative of phenolic acid was also extracted and enriched by the same methodology as the one devised for the extraction of other anti-oxidant molecules, thus yielding a mixture of p-coumaroyl hexose and other polyphenols that seems to be particularly well suited for combatting inflammation, reversing insulin resistance and improving glucose homeostasis in pre-diabetic subjects and thereby prevent progression to type-2 diabetes.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

All publications and patent applications cited in this specification are herein incorporated by reference as if each individual publication or patent application were specifically and individually indicated to be incorporated by reference.

### REFERENCES

1. International Diabetes Federation. IDF Diabetes Atlas. 6th ed. Brussels, Belgium. International Diabetes Federation, 2013
2. Anderson J W, Kendall C W, Jenkins D J. Importance of weight management in type 2 diabetes: Review with meta analysis of clinical studies. J Am Coll Nutr 2003; 22:331-339
3. Jeon C Y, Lokken R P, Hu F B, Van Dam R M. Physical activity of moderate intensity and risk of type 2 diabetes: a systematic review. Diabetes Care 2007; 30:744-752
4. Wellen K E, Hotamisligil G S. Inflammation, stress, and diabetes. J Clin Invest 2005; 115:1111-1119
5. Mursu J, Virtanen J K, Tuomainen T P, Nurmi T, Voutilainen S. Intake of fruit, berries, and vegetables and risk of type 2 diabetes in Finnish men: the Kuopio Ischaemic Heart Disease Risk Factor Study. Am J Clin Nutr 2014; 99:328-333
6. Boeing H, Bechthold A, Bub A, et al. Critical review: vegetables and fruit in the prevention of chronic diseases. Eur J Nutr 2012; 51:637-663
7. Jennings A, Welch A A, Spector T, Macgregor A, Cassidy A. Intakes of anthocyanins and flavones are associated with biomarkers of insulin resistance and inflammation in women. J Nutr 2014; 144:202-208
8. Hanhineva K, Törrönen R, Bondia-Pons I, et al. Impact of dietary polyphenols on carbohydrate metabolism. Int J Mol Sci 2010; 11:1365-1402
9. Denis M C, Desjardins Y, Furtos A, et al. Prevention of oxidative stress, inflammation and mitochondrial dysfunction in the intestine by different cranberry phenolic fractions. Clin Sci (Lond) 2015; 128:197-212
10. Breen D M, Sanli T, Giacca A, Tsiani E. Stimulation of muscle cell glucose uptake by resveratrol through sirtuins and AMPK. Biochem Biophys Res Commun 2008; 374:117-122
11. Nizamutdinova I T, Jin Y C, Chung J I, et al. The anti-diabetic effect of anthocyanins in streptozotocin-induced diabetic rats through glucose transporter 4 regulation and prevention of insulin resistance and pancreatic apoptosis. Mol Nutr Food Res 2009; 53:1419-1429
12. Vidal R, Hernandez-Vallejo S, Pauquai T, et al. Apple procyanidins decrease cholesterol esterification and lipoprotein secretion in Caco-2/TC7 enterocytes. J Lipid Res 2005; 46:258-268
13. Basu A, Lyons T J. Strawberries, blueberries, and cranberries in the metabolic syndrome: clinical perspectives. J Agric Food Chem 2012; 60:5687-5692
14. Takikawa M, Inoue S, Horio F, Tsuda T. Dietary anthocyanin-rich bilberry extract ameliorates hyperglycemia and insulin sensitivity via activation of AMP-activated protein kinase in diabetic mice. J Nutr 2010; 140:527-533
15. De Bock M, Derraik J G, Outfield W S. Polyphenols and glucose homeostasis in humans. J Acad Nutr Diet 2012; 112:808-815
16. Stull A J, Cash K C, Johnson W D, Champagne C M, Cefalu W T. Bioactives in blueberries improve insulin sensitivity in obese, insulin-resistant men and women. J Nutr 2010; 140:1764-1768
17. Hokayem M, Blond E, Vidal H, et al. Grape polyphenols prevent fructose-induced oxidative stress and insulin resistance in first-degree relatives of type 2 diabetic patients. Diabetes Care 2013; 36:1454-1461
18. Scarsella C, Almeras N, Mauriege P, et al. Determination of reference values for fasting insulin levels in a representative sample of the adult Quebec population (Abstract). Atherosclerosis 2000; 151:101
19. Expert Committee on the Diagnosis and Classification of Diabetes Mellitus. Follow-up report on the diagnosis of diabetes mellitus. Diabetes Care 2003; 26:3160-3167
20. Dudonne S, Dube P, Pilon G, et al. Modulation of strawberry/cranberry phenolic compounds glucuronidation by co-supplementation with onion: Characterization of phenolic metabolites in rat plasma using an optimized μSPE-UHPLC-MS/MS method. J Agric Food Chem 2014; 62:3244-3256
21. Labonte M E, Cyr A, Baril-Gravel L, Royer M M, Lamarche B. Validity and reproducibility of a web-based, self-administered food frequency questionnaire. EurJ Clin Nutr 2012; 66:166-173
22. Piche M E, Weisnagel S J, Corneau L, Nadeau A, Bergeron J, Lemieux S. Contribution of abdominal visceral obesity and insulin resistance to the cardiovascular risk profile of postmenopausal women. Diabetes 2005; 54:770-777
23. DeFronzo R A, Tobin J D, Andres R. Glucose clamp technique: a method for quantifying insulin secretion and resistance. Am J Physiol 1979; 237:E214-E223
24. Desbuquois B, Aurbach G D. Use of polyethylene glycol to separate free and antibody-bound peptide hormones in radioimmunoassays. J Clin Endocrinol Metab 1971; 33:732-738
25. Richterich R, Dauwalder H. Determination of plasma glucose by hexokinase-glucose-6-phosphate dehydrogenase method. Schweiz Med Wochenschr 1971; 101:615-618
26. Burstein M, Samaille J. On a rapid determination of the cholesterol bound to the serum alpha- and beta-lipoproteins. Olin Chim Acta 1960; 5:609
27. Moorjani S, Gagne C, Lupien P J, Brun D. Plasma triglycerides related decrease in high-density lipoprotein cholesterol and its association with myocardial infarction in heterozygous familial hypercholesterolemia. Metabolism 1986; 35:311-316
28. Rubio L, Serra A, Chen C Y, et al. Effect of the co-occurring components from olive oil and thyme extracts on the antioxidant status and its bioavailability in an acute ingestion in rats. Food Funct 2014; 5:740-747
29. Kar P, Laight D, Rooprai H K, Shaw K M, Cummings M. Effects of grape seed extract in Type 2 diabetic subjects at high cardiovascular risk: a double blind randomized placebo controlled trial examining metabolic markers, vascular tone, inflammation, oxidative stress and insulin sensitivity. Diabet Med 2009; 26:526-531
30. Basu A, Du M, Leyva M J, et al. Blueberries decrease cardiovascular risk factors in obese men and women with metabolic syndrome. J Nutr 2010; 140:1582-1587
31. Lee I T, Chan Y C, Lin C W, Lee W J, Sheu W H. Effect of cranberry extracts on lipid profiles in subjects with Type 2 diabetes. Diabet Med 2008; 25:1473-1477
32. Hassellund S S, Flaa A, Kjeldsen S E, et al. Effects of anthocyanins on cardiovascular risk factors and inflammation in pre-hypertensive men: a double-blind randomized placebo-controlled crossover study. J Hum Hypertens 2013; 27:100-106
33. Rao S S, Disraeli P, McGregor T. Impaired glucose tolerance and impaired fasting glucose. Am Fam Physician 2004; 69:1961-1968
34. Kahn S E, Prigeon R L, McCulloch D K, et al. Quantification of the relationship between insulin sensitivity and beta-cell function in human subjects. Evidence for a hyperbolic function. Diabetes 1993; 42:1663-1672
35. Pratley R E, Weyer C. Progression from IGT to type 2 diabetes mellitus: the central role of impaired early insulin secretion. Curr Diab Rep 2002; 2:242-248
36. U.S. Departament of Agriculture. USDA Database for the Flavonoid Content of Selected Foods. Beltsville, Md., U.S. Department of Agriculture, 2007
37. Rothwell J A, Perez-Jimenez J, Neveu V, et al. Phenol-Explorer 3.0: a major update of the Phenol-Explorer database to incorporate data on the effects of food processing on polyphenol content. Database (Oxford) 2013; 2013:bat070
38. Brat P, George S, Bellamy A, et al. Daily polyphenol intake in France from fruits and vegetables. J Nutr 2006; 136:2368-2373
39. Anhe G F, Okamoto M M, Kinote A, et al. Quercetin decreases inflammatory response and increases insulin action in skeletal muscle of ob/ob mice and in L6 myotubes. EurJ Pharmacol 2012; 689:285-293
40. Bhattacharya S, Christensen K B, Olsen L C, et al. Bioactive components from flowers of Sambucus nigra L. increase glucose uptake in primary porcine myotube cultures and reduce fat accumulation in Caenorhabditis elegans. J Agric Food Chem 2013; 61:11033-11040
41. Yoon S A, Kang S I, Shin H S, et al. p-Coumaric acid modulates glucose and lipid metabolism via AMP-activated protein kinase in L6 skeletal muscle cells. Biochem Biophys Res Commun 2013; 432:553-557
42. Anhe F F, Roy D, Pilon G, et al. A polyphenol-rich cranberry extract protects from diet-induced obesity, insulin resistance and intestinal inflammation in association with increased Akkermansia spp. population in the gut microbiota of mice. Gut 2014; 0:1-12

1. A strawberry fruit extract comprising at least 500 ppm of p-coumaryl hexose in dry matter.

2. (canceled)

3. The extract of claim 2, comprising a concentration of p-coumaroyl hexose of at least 1000 ppm.

4. (canceled)

5. The extract of claim 4, comprising a concentration of p-coumaroyl hexose of at least 4000 ppm.

6. The extract of claim 1, wherein said strawberry is of the Authentique Orleans variety.

7. A strawberry extract having an ORAC value of at least about 1500 Trolox equivalent (μM/g).

8. The strawberry extract of claim 7, comprising about 5% to about 60% polyphenols.

9. The extract of claim 8, comprising about 30-40% ellagitanins; 20-30 proanthocyanidins; 5 to 10% pelargonidin; and 5-10% quercetin, and 10% to 30% simple phenolics.

10. The extract of claim 9, wherein said simple phenolics comprises at least about 600 ppm of p-coumaryl hexose in dry matter.

11. The extract of claim 1, being in the form of a liquid or a powder.

12. A composition comprising the extract according to claim 1, in admixture with a physiologically acceptable excipient.

13. A capsule or tablet for human oral administration comprising the composition according to claim 12 in the form of a powder or a liquid.

14.-15. (canceled)

16. A process for the enrichment of p-coumaroyl hexose and/or polyphenols from a processed strawberry pulp, the process comprising the steps of:
a) suspending said pulp in a solvent selected from: ethanol, water and aqueous ethanol, at pH between 1.5 and 4.5 to obtain an ethanolic extract;
b) evaporating said solvent and resuspending extract in water to obtain an aqueous extract;
c) submitting said aqueous extract to a hydrophobic chromatography column;
d) eluting said column with aqueous ethanol to obtain a solution enriched in polyphenols.

17.-29. (canceled)

30. A method for the treatment of inflammation in a subject suffering therefrom, comprising the step of administering to said subject an effective amount of a strawberry extract as defined in claim 1.

31. The method of claim 30, wherein said inflammation is selected from: resistance to insulin; diabetes; and metabolic syndrome.

32.-33. (canceled)

34. A method for the management of pre-diabetes in humans, comprising the steps of administering a biologically active amount of the composition of claim 12.

35. A method for the management of pre-diabetes in humans, comprising the steps of administering a biologically active amount of the capsule or tablet according to claim 13.
